(19) United States
(12) Patent Application Publication
(10) Pub. No.: US 2003/0228324 A1
(43) Pub. Date: Dec. 11, 2003

(54) Peptide compositions and methods of producing and using same

(76) Inventors: Andrew J. Malcolm, Edmonton (CA); Rita L. Marcotte, Edmonton (CA); Garry Lund, Edmonton (CA); Robert Hodges, Denver, CO (US)

Correspondence Address: Bell, Boyd & Lloyd LLC, P.O. Box 1135, Chicago, IL, 60690-1135, US

(21) Appl. No.: 10/435,575

(22) Filed: May 9, 2003

Related U.S. Application Data
(63) Continuation-in-part of application No. 10/341,775, filed on Jan. 14, 2003.
Application No. 10/341,775 is a continuation of application No. 09/345,624, filed on Jun. 30, 1999, now Pat. No. 6,541,007.
Application No. 09/345,624 is a continuation of application No. 09/306,241, filed on May 6, 1999, now abandoned.

(51) Int. Cl. A61K 39/00; A61K 39/38

(52) U.S. Cl. 424/184.1

(57) ABSTRACT

Peptide compositions and methods of producing and using same are provided. The peptide compositions are based on pilin peptides derived from Pseudomonas aeruginosa. The compositions include antigens and antibodies immunoreactive against the antigens. The compositions can be utilized in vaccines for treatment purposes, such as to treat or prevent infection associated with Pseudomonas aeruginosa and/or other infectious agents. Further, the compositions can be utilized to produce antibody or monoclonal antibody therapeutic to treat or prevent infection associated with Pseudomonas aeruginosa and/or other infectious agents.

### CROSS REFERENCE TO RELATED APPLICATIONS

This patent application is a continuation-in-part of U.S. patent application Ser. No. 10/341,775 filed on Jan. 14, 2003 which is a continuation of U.S. patent application Ser. No. 09/345,624, filed on Jun. 30, 1999 which issued as U.S. Pat. No. 6,541,007 and is a continuation of U.S. patent application Ser. No. 09/306,241, filed on May 6, 1999, now abandoned, the disclosures of which are incorporated herein by reference.

### BACKGROUND OF THE INVENTION

The present invention generally relates to peptide compositions. More specifically, the present invention relates to peptide compositions based on pilin peptides derived from Pseudomonas aeruginosa, and includes antigens, antibodies, and methods of producing and using same.

Pseudomonas aeruginosa is a serious opportunistic gram-negative bacterial pathogen, which can cause fatal infections in immunocompromised and immunosuppressed patients. See, Irvin, R. T., “Attachment and colonization of Pseudomonas aeruginosa: Role of the surface structures”, in PSEUDOMONAS AERUGINOSA AS AN OPPORTUNISTIC PATHOGEN, (Campa, M., M. Bendinelli, and H. Friedman, Eds.), pp 19-42, Plenum Press, New York (1993); Pier, G. B., J. Infect. Dis. 151:575-580 (1985); Rivera, M. and Nicotra, M. B., Am. Rev. Respir. Dis. 126:833-836 (1982); and Todd, T. R. J., et al., Am. Rev. Respir. Dis. 140:1585-1589 (1989).

The first step in the infection process is the attachment to the host cell. This attachment is mediated by pili on the surface of the bacterium. See, Pier, G. B., J. Infect. Dis. 151:575-580 (1985); Irvin, R. T., et al., Infect. Immun. 57:3720-3726 (1989); and Lee, K. K., et al., Mol. Microbiol. 3:1493-1499 (1989). Pseudomonas aeruginosa uses several adhesins to mediate attachment to mucosal surfaces, but analysis of the binding properties of the adhesins and binding competition studies indicate that the pilus is the dominant adhesin responsible for initiating infections. See, Irvin, R. T., “Attachment and colonization of Pseudomonas aeruginosa: Role of the surface structures”, in PSEUDOMONAS AERUGINOSA AS AN OPPORTUNISTIC PATHOGEN, (Campa, M., M. Bendinelli, and H. Friedman, Eds.), pp 19-42, Plenum Press, New York (1993); Doig, P., et al., Infect. Immun. 55:1517-1522 (1987); McEachran, D. and Irvin, R. T., Can. J. Microbiol. 31:563-569 (1985); and Irvin, R. T., et al., Microb. Ecol. Health Dis. 3:39-47 (1990).

Pseudomonas aeruginosa pili are polarly located, with a structure resembling a hollow tube of 5.2 nm in outer diameter, 1.2 nm in central channel diameter, and an average length of 2.5 μm. See, Bradley, D. E., Genet. Res. 19:39-51 (1972); Folkhard, W. F., et al., J. Mol. Biol. 149:79-93 (1981); and Paranchych, W., et al., Clin. Invest. Med. 9:113-118 (1986).

The pilus of Pseudomonas aeruginosa is composed of multiple copies of a 13-17 kDa monomeric protein subunit called pilin. The C-terminal region of the pilin monomer contains the epithelial cell binding domain and is semiconserved in seven different strains of this bacterium. See, Irvin, R. T., et al., Infect. Immun. 57:3720-3726 (1989); Paranchych, W., et al., Clin. Invest. Med. 9:113-118 (1986); Paranchych, W., et al., “Expression, processing, and assembly of Pseudomonas aeruginosa N-methylphenylalanine pilin”, in PSEUDOMONAS: BIOTRANSFORMATIONS, PATHOGENESIS AND EVOLVING BIOTECHNOLOGY, (Sliver, S., et al., Eds.), pp 343-351, American Society for Microbiology, Washington, D.C. (1990); and Pasloske, B. L., et al., J. Bacteriol. 170:3738-3741 (1988).

This semiconserved region has also been shown to bind to a minimal structural carbohydrate receptor sequence, β-GalNAc(1-4)βGal, found in glycosphingolipids, specifically asialo-GM1 and asialo-GM2. See, Yu, L., et al., Infect. Immun. 62:5213-9 (1994); and Sheth, H. B., et al., Mol. Microbiol. 11:715-23 (1994). Furthermore, the C-terminal disulfide-bridged 17-residue region of the PAK pilin is known to be important in raising antibodies that block binding of both bacteria or their pili to epithelial cells. See, Lee, K. K., et al., Mol. Microbiol. 3:1493-1499 (1989); Doig, P., et al., Infect. Immun. 58:124-130 (1990); and Lee, K. K., Infect. Immun. 57:520-526 (1989). Both monoclonal antisera generated from Pseudomonas aeruginosa pili or polyclonal antisera generated from synthetic peptides representing the receptor binding domain of the pathogen have been shown to be efficacious in preventing infection. See, Sheth, H. B., et al., Biomed. Pept. Proteins and Nucleic Acids 1:141-148 (1995).

Different types of Pseudomonas aeruginosa immunogens have been tried or are under development as vaccines. These include lipopolysaccharides (See, Hanessian, S., et al., Nature (London) 229:209-210 (1971); and MacIntyre, S., et al., Infect. Immun. 52 (1986)), polysaccharide (See, Pier, G. B. and Thomas, D. M., J. Infect. Dis. 148:206-213 (1983)), polysaccharide conjugate (See, Cryz, S. J., et al., Antibiot. Chemother. 42:177-183 (1989)), outer-membrane protein (See, Lam, J. S., et al., Infect. Immun. 42:88-98 (1983); and Matthews-Greer, J. M. and Gilleland, Jr., H. E., J. Infect. Dis. 155:1281-1291(1987)), mucoid exopolysaccharide (See, Pier, G. B., et al., Science 249:537-540 (1990)), flagella (See, Holder, I. A. and Naglich, J. G., J. Trauma. 26:118-122 (1986); and Rotering, H. and Dorner, F., Antibiot. Chemother. 42:218-228 (1989)), protease (See, Sezen, I. Y., et al., Zentralbl. Bakteriol. Hyg. I. Abt. Orig. 231:126-132 (1975)), elastase (See, Cryz, S. J., Jr., et al., Infect. Immun. 39:1072-1079 (1983)), exotoxin A (See, Cryz, S. J., Jr., et al., Infect. Immun. 39:1072-1079 (1983); and Lydick, E., et al., J. Infect. Dis. 151:375 (1985)), and lipoprotein I (See, Finke, M., et al., Infect. Immun. 59:1251-1254 (1991)).

An alternate to these approaches to vaccination against Pseudomonas aeruginosa could employ a multivalent pili vaccine. However, a potential problem exists in this approach: inhibition of the immune response to one antigen or determinant by the administration of another antigen or determinant. This phenomenon, termed antigenic competition, leads to the reduction of antibody production and has been shown to occur between chemically related and unrelated antigens and also between associated and non-associated antigenic determinants. See, Taussig, M. J., “Antigenic competition”, in THE ANTIGENS (Sela, M., Ed.), pp 333-368, Academic Press, New York (1977). An example of this type of competition has been reported by Hunt and coworkers in the development of a multivalent pili vaccine against ovine footrot. See, Hunt, J. D., et al., Vaccine 13:1649-1657 (1995); and Hunt, J. D., et al., Immunol. Cell Biol. 74:81-89 (1996). In this case, antigenic competition occurs between the nine pili serotypes of the bacterium Dichelobacter nodosus that are required in a vaccine for complete protection against the disease. These results suggested that a cocktail or multicomponent vaccine composed of synthetic peptide immunogens representing the known strains of Pseudomonas aeruginosa pili may be problematic.

A need therefore exists to provide improved peptide compositions and methods of producing and using same.

### SUMMARY OF THE INVENTION

The present invention relates to peptide compositions that can be used in a variety of different applications, such as therapeutic and diagnostic. More specifically, the peptide compositions of the present invention are based on pilin peptides derived from Pseudomonas aeruginosa. The compositions include antigens and antibodies immunoreactive against the antigens. The compositions can be utilized in vaccines for treatment purposes, such as to treat or prevent infection associated with Pseudomonas aeruginosa and/or other infectious organisms.

In an embodiment, the present invention includes a peptide vaccine for immunizing or treating a patient for infection by a Pseudomonas aeruginosa (PA) infection. The invention includes (i) the peptide identified as SEQ ID NOS. 3-6; and (ii) a carrier protein conjugated to the peptide. The peptide vaccine is useful in protecting a subject against Pseudomonas aeruginosa infection, by administering the vaccine to the subject, also in accordance with the invention.

In another embodiment, the present invention includes a C-terminal PA pilin peptide having the amino acid sequence identified as SEQ ID NO: 3, and analogs thereof having one of residues T, K, or A at position 130, D, T, or N at position 132, Q, A, or V at position 133, E, P, N, or A at position 135, Q, M, or K at position 136, and I, T, L, or R at position 138, excluding SEQ ID NOS: 1, 2, 9, 10, and 11. The claimed peptide is also characterized by its ability to cross-react with antibodies against the corresponding C-terminal peptides from PA strains PAK and PAO, preferably also against antibodies specific against a C-terminal peptide from PA strains CD4, K122, or KB7.

In yet another embodiment, the present invention includes a method of selecting a peptide for use in a vaccine against Pseudomonas aeruginosa. The method includes the steps of (i) constructing a library of 1296 C-terminal peptides having the amino acid sequence identified as SEQ ID NO: 3, and analogs thereof having one of residues T, K, or A at position 130, D, T, or N at position 132, Q, A, or V at position 133, E, P, N, or A at position 135, Q, M, or K at position 136, and I, T, L, or R at position 138, and (ii) selecting library members which are cross-reactive with antibodies against the corresponding C-terminal peptides from PA strains PAK, PAO and the like.

In a further embodiment, an antibody is provided. The antibody in an embodiment is produced against a peptide composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

In an embodiment, the antibody includes a monoclonal antibody.

In an embodiment, the antibody includes a humanized monoclonal antibody.

In an embodiment, the humanized monoclonal antibody includes a human monoclonal antibody.

In an embodiment, the antibody includes a mouse monoclonal antibody.

In an embodiment, a cell line produces the antibody.

In an embodiment, the peptide composition further includes a carrier molecule coupled to the peptide.

In an embodiment, the carrier molecule includes a carrier protein, a carrier glycoprotein, a carrier carbohydrate, tetanus toxoid/toxin, diphtheria toxoid/toxin, Pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), commensal bacteria, Phage, peptides attached to recombinant IgG1, carrier sub-units thereof, combinations thereof and the like.

In an embodiment, the antibody is isolated or purified.

In an embodiment, the antibody includes an isolated or a purified antibody or fragment thereof that can selectively bind to an epitope associated with a bacterial strain including, for example, Pseudomonas aeruginosa, Acinetobacter spp. Burkholderia cepacia, Haemophilus influenza and Pasteurella multiocida.

In an embodiment, the antibody or fragment thereof is capable of inhibiting binding of an infectious agent including Pseudomonas aeruginosa to a host cell.

In yet a further embodiment, the present invention provides a humanized monoclonal antibody or fragment thereof that is immunoreactive with Pseudomonas aeruginosa pilus protein.

In still yet a further embodiment, the present invention provides a humanized monoclonal antibody or fragment thereof that is immunoreactive with a C-terminal disulfide-linked peptide region of Pseudomonas aeruginosa pilus protein.

In another embodiment, the present invention provides a humanized monoclonal antibody produced against a peptide composition including a peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

In a further embodiment, the present invention provides a purified antibody or fragment thereof that binds to an epitope in a Pseudomonas aeruginosa pilin peptide or variant thereof selected from the group consisting of SEQ ID NO: 3, SEQ ID NO: 4, SEQ ID NO: 5, SEQ ID NO: 6, SEQ ID NO: 7 and SEQ ID NO: 8.

In yet a further embodiment, the present invention provides a humanized antibody a fragment thereof that binds to an epitope in a Pseudomonas aeruginosa pilin peptide or variant thereof selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

In yet another embodiment, the present invention provides a pharmaceutical agent. The agent includes an antibody or fragment thereof produced against a composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

In still yet another embodiment, the present invention provides a method of producing an antibody. The method includes the steps of providing a peptide composition including a peptide selected from the group consisting of SEQ ID NO: 3, SEQ ID NO: 4, SEQ ID NO: 5, SEQ ID NO: 6, SEQ ID NO: 7 and SEQ ID NO: 8; and producing the antibody against the peptide composition.

In a further embodiment, the present invention provides a method for producing a humanized monoclonal antibody. The method includes the steps of providing a peptide composition including a peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14; and administering to an animal the peptide to produce the antibody.

In a still further embodiment, the present invention provides a method of making an antibody including immunizing a non-human animal with an immunogenic peptide composition wherein the composition includes a peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

In yet another embodiment, the present invention provides a method of treating or preventing infection by an infectious organism including Pseudomonas aeruginosa. The method includes administering a pharmaceutical agent including an antibody or fragment thereof produced against a composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

In still yet another embodiment, the present invention provides a method of treating or immunizing a subject against infection by an infectious agent including Pseudomonas aeruginosa. The method includes administering a pharmaceutical agent including an antibody or fragment thereof produced against a composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

An advantage of the present invention is to provide improved peptide compositions.

Another advantage of the present invention is to provide improved peptide compositions based on pilin peptides derived from Pseudomonas aeruginosa.

Yet another advantage of the present invention is to provide methods of producing improved peptide compositions.

Yet still another embodiment of the present invention is to utilize the improved peptide compositions for therapeutic purposes.

A further embodiment of the present invention is to utilize the improved peptide compositions for diagnostic purposes.

Additional features and advantages of the present invention are described in, and will be apparent from, the following Detailed Description of the Invention and the figures.

### BRIEF DESCRIPTION OF THE FIGURES

FIG. 1 shows the native sequences PAK (SEQ ID NO: 1) and PAO (SEQ ID NO: 2), and the sequences of six PAK-peptide analogs, identified as CS1 (SEQ ID NO: 3); CS2 (SEQ ID NO: 4); CS3 (SEQ ID NO: 5); CS4 (SEQ ID NO: 6); E135A (SEQ ID NO: 7); and E135P (SEQ ID NO: 8), where the amino acid variations with the PAK-peptide sequence in the six analogs are indicated by circles.

FIG. 2 shows survival times of animals immunized with the E135A antigen (SEQ ID NO: 7), after challenge with PAO wild-type.

FIG. 3 shows survival times of animals immunized with the CS2 (double mutant) (SEQ ID NO: 4), PAO (SEQ ID NO: 2) or PAK (SEQ ID NO: 1) peptide, after challenge with PAO wild-type.

FIG. 4 shows survival times of animals immunized with the CS1 (SEQ ID NO: 3), CS2 (SEQ ID NO: 4), or CS3 (SEQ ID NO: 5) peptide, after challenge with P1 wild-type.

FIG. 5 shows survival times of animals immunized with the CS4 (SEQ ID NO: 6) peptide, after challenge with P1 wild-type.

FIG. 6 shows survival times of animals immunized with the CS1 (SEQ ID NO: 3), CS2 (SEQ ID NO: 4), or EXO-S peptide, after challenge with KB7 wild-type.

FIG. 7 shows survival times of animals immunized with the CS2 (SEQ ID NO: 4), PAO (SE ID NO: 2) or PAK (SEQ ID NO: 1) peptide, after challenge with PAK wild-type.

FIG. 8 are competition ELISA plots of the native peptides (PAK, PAO, KB7 and P1) required to achieve 50% inhibition of antibody binding to PAK pili in the presence of antisera E135A, CS1, and PAK, where the open columns represent the peptide antigens (E135A, CS1, and PAK) to which the antisera were generated.

FIG. 9 shows consensus and amino acid variations among in the C-terminal region peptides in five strains of PA.

FIG. 10 shows the sequences of the C-terminal cell surface binding domains of three PA strains P1, 492C, and TBOU1.

FIG. 11 shows the sequences of the C-terminal cell surface binding domains of four Pseudomonas aeruginosa pilin strains PAK, PAO, KB7 and P1 together with the sequences of three PAK analogues I138A, E135P and E135A.

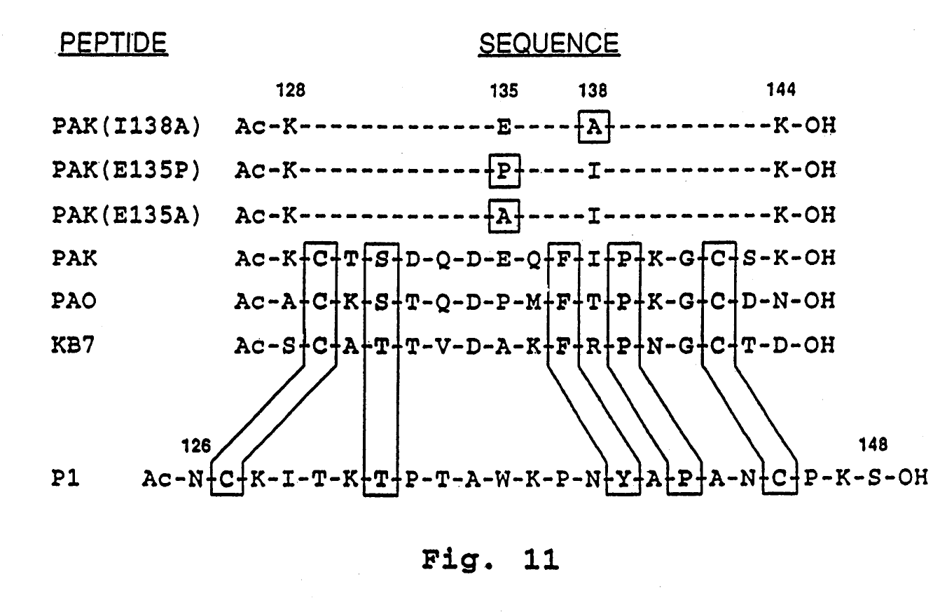

FIG. 12 are competition ELISA plots of the native peptides (PAK, PAO, KB7 and P1) required to achieve 50% inhibition of antibody binding to PAK pili in presence of 17-R1, 17-O1, E135A, E135P and I138A, where the open columns represent the peptide antigens (I138A, E135P and E135A) to which the antisera I138A, E135P and E135A were generated.

FIG. 13 represents a Western Blot Analysis of CS1 monoclonal antibodies made in accordance with an embodiment of the present invention with respect to recombinant pilin protein.

FIG. 14 represents a Western Blot Analysis of CS1 monoclonal antibodies made in accordance with an embodiment of the present invention with respect to various bacterial cell extracts.

### DETAILED DESCRIPTION OF THE INVENTION

The present invention generally relates to peptide compositions. In particular, the peptide compositions of the present invention are based on pilin peptide derived from Pseudomonas aeruginosa. The compositions include peptide antigens and antibodies or fragments thereof immunoreactive against the antigens. The compositions can be utilized in vaccines for treatment purposes, such as to treat or prevent infection associated with Pseudomonas aeruginosa and/or other infectious organisms. It should be appreciated that the compositions of the present invention can be utilized in a variety of suitable applications including therapeutic and diagnostic.

In an embodiment, the peptide composition includes the SEQ ID NOS: 1-14 (See Below) can be used to elicit an immune response in an animal for at least two purposes. Where the composition acts as a vaccine by eliciting an immune response in the animal, the resulting antibodies or T-cell mediated immunity can protect the animal from a subsequent attack involving the same epitopes (active immunity). Alternatively, the composition can be used to produce antibodies, which can be used as a research tool, or administered to a second animal to protect the second animal from a subsequent attack involving the same epitopes (passive immunity).

Peptide SEQ ID NOS. 1-14

| | | |
|---|---|---|
| Sequence ID | Strain | Sequence |

| | | |
|---|---|---|
| 1 | PAK | KCTSDQDEQFIPKGCSK |
| 2 | PAO | ACKSTQDPMFTPKGCDN |
| 3 | CS1 | KCKSDQDPQFIPKGCSK |
| 4 | CS2 | KCTSTQDPQFIPKGCSK |
| 5 | CS3 | KCTSTQDPQFTPKGCSK |
| 6 | CS4 | KCKSTQDEMFTPKGCSK |
| 7 | E135A | KCTSDQDAQFIPKGCSK |
| 8 | E135P | KCTSDQDPQFIPKGCSK |
| 9 | CD4 | TCTSTQEEMFIPKGCNK |
| 10 | K122-4 | ACTSNADNKYLPKTCQT |
| 11 | KB7 | SCATTVDAKFRPNGCTD |
| 12 | P1 | NCKITKTPTAWKPNYAPANCPK |
| 13 | 492 | TCGITGSPTNWKANYAPANCPK |
| 14 | TBOU1 | GCSISSTPANWKPNYAPSNCPK |

Based on standard peptide nomenclature, abbreviations for amino acid residues that have been used herein are shown in the Table below:

Symbol

| 1 Letter | 3 Letter | Amino Acid |
|---|---|---|
| Y | TYR | -L-tyrosine |
| G | GLY | -glycine |
| F | PHE | -L-phenylalanine |
| M | MET | -L-methionine |
| A | ALA | -L-alanine |
| S | SER | -L-serine |
| I | ILE | -L-isoleucine |
| L | LEU | -L-leucine |
| T | THR | -L-threonine |
| V | VAL | -L-valine |
| P | PRO | -L-proline |
| K | LYS | -L-lysine |
| N | ASN | -L-asparagine |
| H | HIS | -L-histidine |
| Q | GLN | -L-glutamine |
| E | GLU | -L-glutamic acid |
| W | TRP | -L-tryptophan |
| R | ARG | -L-arginine |
| D | ASP | -L-aspartic acid |
| C | CYS | -L-cysteine |

To augment the immune response elicited, it may be preferable to couple the peptides of SEQ ID NOS: 1-14 to any carrier molecule or carrier proteins. Various protein, glycoprotein, carbohydrate or sub-unit carriers can be used, including but not limited to, tetanus toxoid/toxin, diphtheria toxoid/toxin, pseudomonas mutant carrier, bacteria outer membrane proteins, crystalline bacterial cell surface layers, various endo or exotoxins, serum albumin, gamma globulin or keyhole limpet hemocyanin, recombinant, exotoxin A, LT toxin, Cholera B toxin, Klebsiella pneumoniae OmpA, Bacterial flagella, Clostridium difficile recombinant toxin A, Peptide dendrimers (multiple antigenic peptides), pan DR epitope (PADRE), Commensal bacteria, Phage (displaying peptide on and bacteria phages), attachment of peptides to recombinant IgG1 and/or other suitable constituents.

In addition, the peptides of SEQ ID NOS: 1-14 or their conjugates with carrier proteins may be further mixed with adjuvants to elicit an immune response, as adjuvants may increase immunoprotective antibody titers or cell mediated immunity response. Such adjuvants can include, but are not limited to, MPL+TDM+CWS (SIGMA), MF59 (an oil-in-water emulsion that includes 5% squalene, 0.5% sorbitan monoleate and 0.5% sorbitan trioleate Chiron), Heat—labile toxin (HLT), CRM₁₉₇ (nontoxic genetic mutant of diphtheria toxin), Squalene (IDEC PHARMACEUTICALS CORP.), Ovalbumin (SIGMA), Quil A (SARGEANT, INC.), Aluminum phosphate gel (SUPERFOS BIOSECTOR), Cholera holotoxin (CT LIST BIOLOGICAL LAB.), Cholera toxin B subunit (CTB), Cholera toxin A subunit—Protein A D—fragment fusion protein, Muramyl dipeptide (MDP), Adjumera (polyphosphazene, VIRUS RESEARCH INSTITUTE), SPT (an emulsion of 5% squalene, 0.2% Tween 80, 1.25% Pluronic L121 with phosphate—buffered saline ph 7.4), Avridine® (M6 PHARMACEUTICALS), Bay R1005 (BAYER), Calcitrol (SIGMA), Calcium phosphate gel (SARGEANT INC.), CRL 1005 (Block co-polymer P1205, VAXCEL CORP.), DHEA (MERCK), DMPC (GENZYME PHARMACEUTICALS and FINE CHEMICALS). DMPG (GENZYME PHARMACEUTICALS and FINE CHEMICALS), Gamma Inulin, Gerbu Adjuvant (CC BIOTECH CORP.), GM-CSF, (IMMUNEX CORP.), GMDP (PEPTECH LIMITED), Imiquimod (3M PHARMACEUTICALS), ImmTher™ (ENDOREX CORPORATION), ISCOM™ (ISCOTEC AB), Iscoprep 7.0.3™ (ISCOTEC AB), Loxoribine, LT—Oral Adjuvant (E. coli labile enterotoxin, protoxin, BERNA PRODUCTS CORP.), MTP-PE (CIBA-GEIGY LTD), Murametide, (VACSYN S.A.), Murapalmitine (VACSYN S. A.), Pluronic L121 (IDEC PHARMACEUTICALS CORP.), PMMA (INSTITUT FÜR PHARMAZEUTISCHE TECHNOLOGIE), SAF-1 (SYNTEX ADJUVANT FORMULATION CHIRON), Stearyl tyrosine (BIOCHEM THERAPEUTIC INC.), Theramidea (IMMUNO THERAPEUTICS INC.), Threonyl—MDP (CHIRON), FREUNDS complete adjuvant, FREUNDS incomplete adjuvant, aluminum hydroxide, dimethyldioctadecyl-ammonium bromide, Adjuvax (ALPHA-BETA TECHNOLOGY), Inject Alum (PIERCE), Monophosphoryl Lipid A (RIBI IMMUNOCHEM RESEARCH), MPL+TDM (RIBI IMMUNOCHEM RESEARCH), Titermax (CYTRX), QS21, t Ribi Adjuvant System, TiterMaxGold, QS21, Adjumer, Calcitrol, CTB, LT (E. coli toxin), LPS (lipopolysaccharide), Avridine, the CpG sequences (Singh et al., 1999 Singh, M. and Hagum, D., Nature Biotechnology 1999 17:1075-81) toxins, toxoids, glycoproteins, lipids, glycolipids, bacterial cell walls, subunits (bacterial or viral), carbohydrate moieties (mono-, di-, tri-, tetra-, oligo- and polysaccharide), various liposome formulations or saponins. Combinations of various adjuvants may be used with the antigen to prepare the immunogen formulations. Adjuvants administered parentally or for the induction of mucosal immunity may be used.

The composition can be administered by various delivery methods including intravascularly, intraperitoneally, intramuscularly, intradermally, subcutaneously, orally, nasally or by inhalation. In an embodiment, the compositions can further include a pharmaceutically acceptable excipient and/or carrier. Such compositions are useful for immunizing any animal, which is capable of initiating an immune response, such as primate, rodent, bovine, ovine, caprine, equine, leporine, porcine, canine or avian species. Both domestic and wild animals may be immunized. The exact formulation of the compositions will depend on the particular peptide or peptide-carrier conjugate, the species to be immunized, and the route of administration.

As previously discussed, the antibodies or fragment thereof produced against SEQ ID NOS: 1-14 can be included in a pharmaceutical composition and administered to an animal. In an embodiment, the pharmaceutical composition includes a pharmaceutically acceptable carrier, and optionally can include pharmaceutically acceptable excipients. The pharmaceutical composition can be administered intravascularly, intraperitoneally, intramuscularly, intradermally, subcutaneously, orally, nasally or by aerosol inhalation. Preferably the pharmaceutical composition is administered intravascularly, intramuscularly, orally, nasally or by aerosol inhalation.

In an embodiment, the present invention includes antibodies, particularly monoclonal antibodies, which are derived from the antibodies produced against a peptide of SEQ ID NOS: 1-14. In particular, hybridomas can be generated using a peptide of SEQ ID NOS: 1-14 and recombinant derivative antibodies can be made using these hybridomas according to well-known genetic engineering methods. See, Winter, G., and Milstin C., Nature, 1991, 349 293-299, herein incorporated by reference. For example, the DNA fragment coding for the variable regions of the monoclonal antibodies can be obtained by polymerase chain reactions (PCR). The PCR primers can be oligonucleotides, which are complementary to the constant regions of the heavy chain or light chain, and the PCR template can be the total cDNA or genomic DNA prepared from the hybridomas. Alternatively, a cDNA library can be prepared from the hybridomas and screened with probes which correspond to the constant regions of immunoglobulin heavy chain or light chain to obtain clones to the heavy chain or light chain produced by the particular hybridoma.

Subsequently, the DNA fragment for the variable regions can be inserted into an expression vector and joined in frame with the cDNA sequences of a selected constant region. The constant region can be the human constant sequence to make humanized antibodies, the goat constant sequences to make goat antibodies, the IgE constant sequences to make IgE which recognized the peptide of formula I, and the like. Thus, antibodies with the same antigen recognition ability but different constant regions can be produced. Of particular interest are humanized antibodies, which can be used as therapeutic agents against a disease associated with the cognate antigen in humans without eliciting an undesired immune response against the humanized constant region.

Other methods known in the art to humanize antibodies or produce humanized antibodies can be utilized as well and are herein incorporated by reference. These methods can include but are not limited to the xenomouse technology developed by ABGENIX INC. (See, U.S. Pat. Nos. 6,075,181 and 6,150,584) and the methods developed by BIOVATION, BIOINVENT INTERNATIONAL AB, PROTEIN DESIGN LABS, APPLIED MOLECULAR EVOLUTION, INC., IMMGENICS PHARMACEUTICALS INC., MEDAREX INC., CAMBRIDGE ANTIBODY TECHNOLOGY, ELAN, EOS BIOTECHNOLOGY, MEDIMMUNE, MORPHOSYS, UROGENSYS INC., AVANIR PHARMACEUTICAL/XENEREX BIOSCIENCES, AFFIBODY AB, ALLEXION ANTIBODY TECHNOLOGIES, ARIUS RESEARCH INC., CELL TECH, XOMA, IDEC PHARMACEUTICALS, NEUGENESIS, EPICYTE, SEMBIOSYS GENETICS INC., BIOPROTEIN, GENZYME THERAPEUTICS, KIRIN, GEMINI SCIENCES, HEMATECH. Likewise, other methods known in the art to screen human antibody secreting cells to SEQ ID NOS: 1-14 peptide antigens can be also be utilized.

As used herein, the term “humanized antibody” or other like terms means an antibody that includes a human protein sequence in at least a portion thereof. The amount of human protein sequence can vary depending on how the antibody is made. A “fully humanized antibody” or “human antibody” as the terms or like terms are used herein can be made, for example, with xenomouse technology as discussed above.

The formulation for the composition, including either SEQ ID NO: 1-14 peptide or an antibody against SEQ ID NOS: 1-14 peptides will vary depending on factors, such as the administration route, the size and species of the animal to be administered, and the purpose of the administration. Suitable formulations for use in the present invention can be found in Remington's Pharmaceutical Sciences.

The conjugates according to an embodiment of the present invention can be used as classical vaccines or immunogens, which elicit specific antibody production or stimulate specific cell mediated immunity responses. They may also be utilized as therapeutic modalities, for example, to stimulate the immune system to recognize tumor-associated antigens; as immunomodulators, for example, to stimulate lymphokine/cytokine production by activating specific cell receptors; as prophylactic agents, for example, to block receptors on cell membrane preventing cell adhesion; as diagnostic agents, for example, to identify specific cells; and as development and/or research tools, for example, to stimulate cells for monoclonal antibody production.

The following examples are offered to illustrate this invention and are not to be construed in any way as limiting the scope of the present invention.

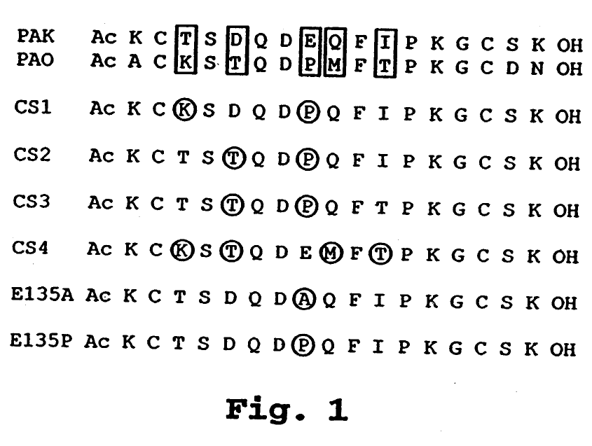

Initially, the cross-reactivity of the native sequence PAK (FIG. 1; SEQ ID NO: 1) when it was used as immunogens in rabbits was examined. Polyclonal serum from this immunogen was studied using competition ELISA. The results demonstrated that this antiserum was cross-reactive with the native PAO sequence (SEQ ID NO: 2) in the presence of native PAK sequence on plates coated with PAK strain pili. This cross-reactivity did not extend to other strains tested.

Initially, position 135 in the PAK sequence was chosen as a mutation site for increasing cross-reactivity, and proline from the homologous position in the native PAO sequence was used at position 135 in the native PAK backbone (FIG. 1, E135P, SEQ ID NO: 8). Competition results indicated that the antiserum raised to this immunogen was less cross-reactive than native PAK antiserum and was not cross-reactive with any of the other strains tested (i.e. KB7 and P1).

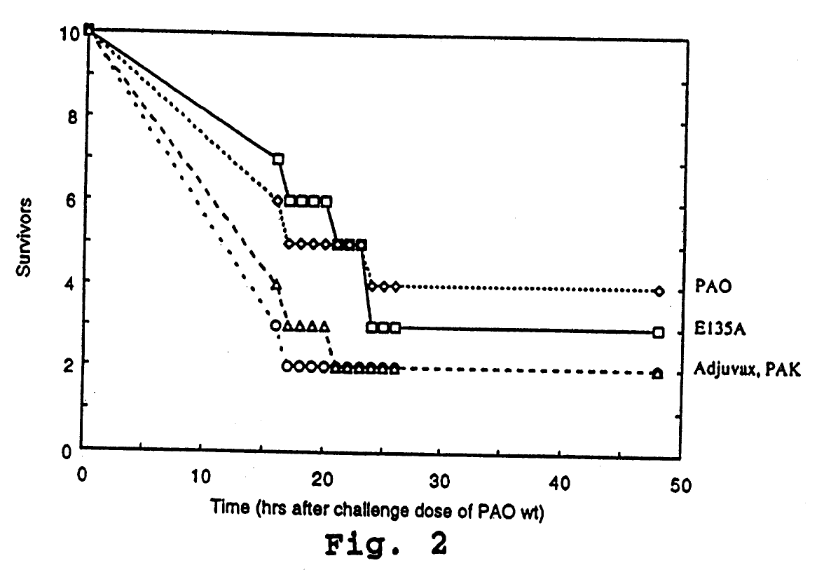

A second single mutant sequence was also constructed, which contained alanine at position 135 (FIG. 1; E135A, SEQ ID NO: 7). This mutant contained the homologous residue, alanine, from position 135 in strain KB7 at position 135 in the PAK backbone. This sequence was found to generate antiserum which was as cross-reactive as native PAK sequence with PAO. Furthermore, this cross-reactivity was more broadly based than that of native PAK sequence; cross-reactivity was found against strains PAO, KB7 and P1. In PAOwt challenge experiments (FIG. 2), E135A immunization demonstrated enhanced survival time in a mouse model.

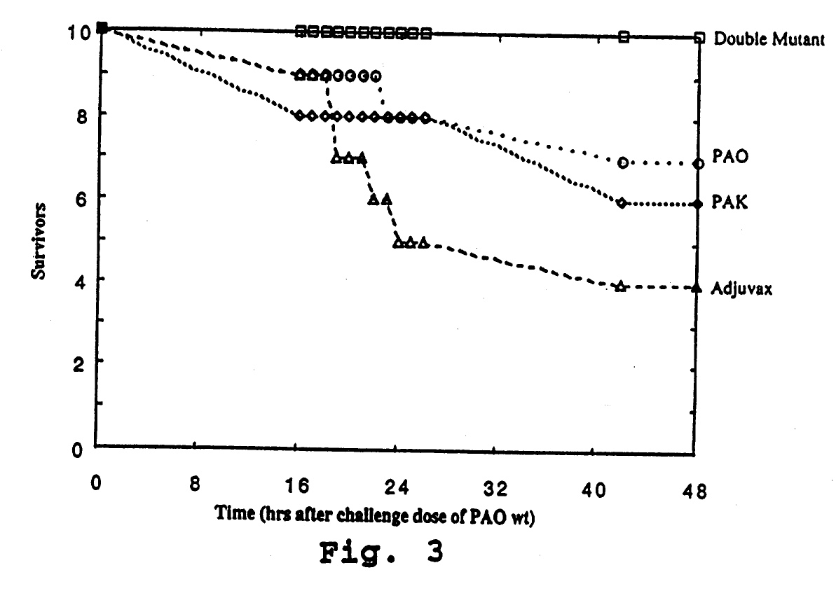

A double mutant (FIG. 1; CS2, SEQ ID NO: 4) (D132T, E135P)) was also synthesized. Results obtained from competition ELISA demonstrate that polyclonal antiserum raised in rabbits to the double mutant had enhanced cross-reactivity to PAO over that demonstrated by PAK. Furthermore, PAOwt challenge experiments demonstrated (FIG. 3) complete protection against challenge with PAOwt.

FIG. 1 shows the native sequences PAK and PAO (residues 128-144), the single mutant sequences E135A, E135P and the multiple mutations indicated as CS1 (SEQ ID NO: 3, CS2 (SEQ ID NO: 4, CS3 (SEQ ID NO: 5), and CS4 (SEQ ID NO: 6). The figure is designed to highlight the differences between the various sequences (boxed residues). For instance, there are eight differences between the native sequences PAK and PAO. There is only one difference between PAK and E135A and eight differences between E135A and PAO, yet the peptide E135A generates antiserum, which shows enhanced cross-reactivity to PAO and enhanced survival time in challenge from PAOwt (FIG. 2). In contrast, E135P has seven differences compared to PAO and only one difference compared to PAK, yet the peptide generates antiserum, which is strain specific for PAK and is less cross-reactive to PAO than PAK peptide. The double mutant CS2 has six differences with respect to PAO and two differences with respect to PAK. The antiserum provides complete protection to challenge from PAOwt (FIG. 3).

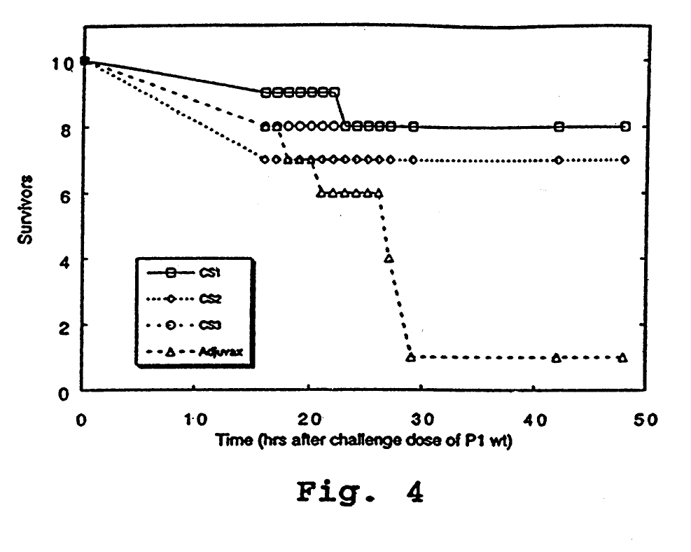

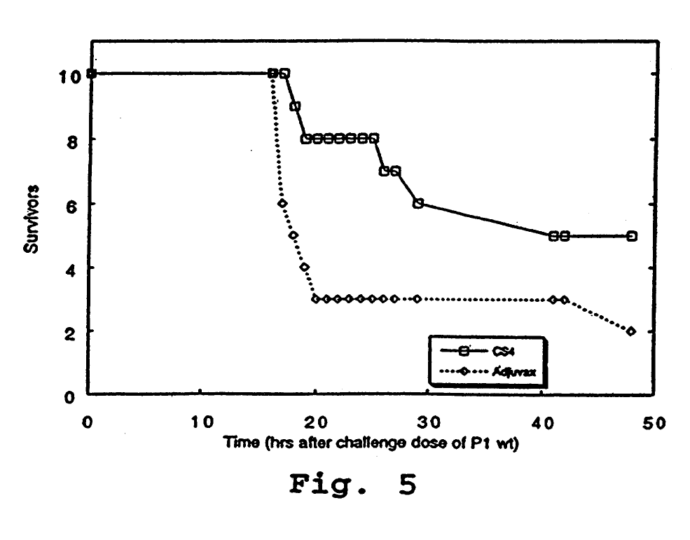

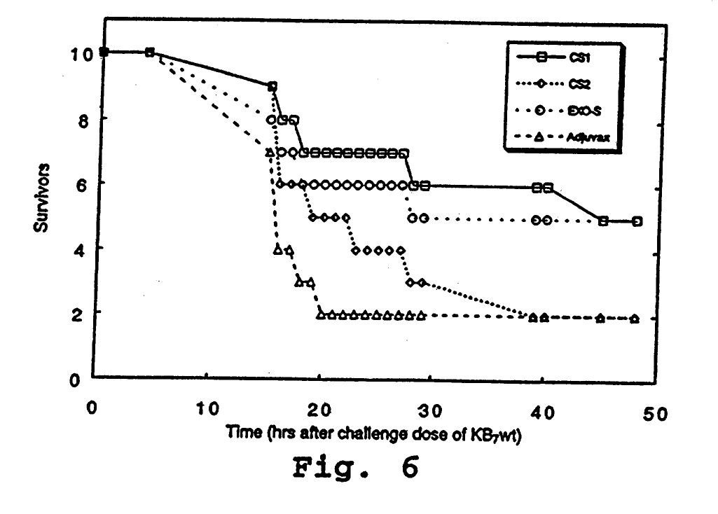

Similar vaccination methods demonstrated that CS1, CS2, and CS3 all provided a high level of protection against infection in animals challenged with P1 (FIG. 4); that CS4 provided good protection against animals also challenged with P1 (FIG. 5); that CS1 provided good protection against infection in animals challenged with KB7, (FIG. 6); and that CS2 provided a very high level of protection in animals challenged with PAK.

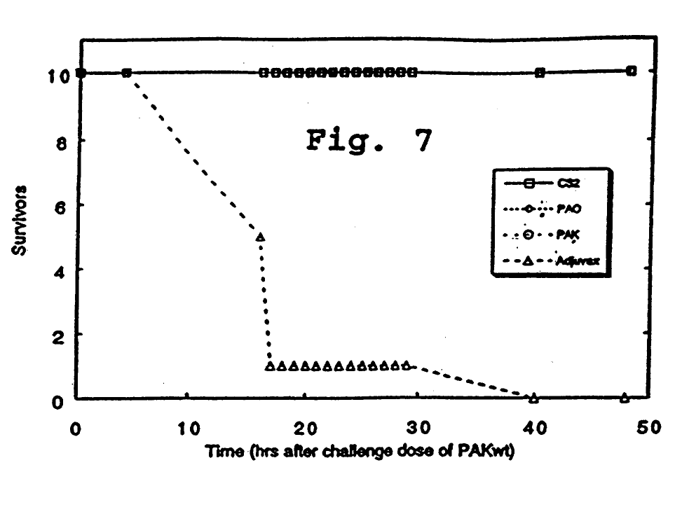

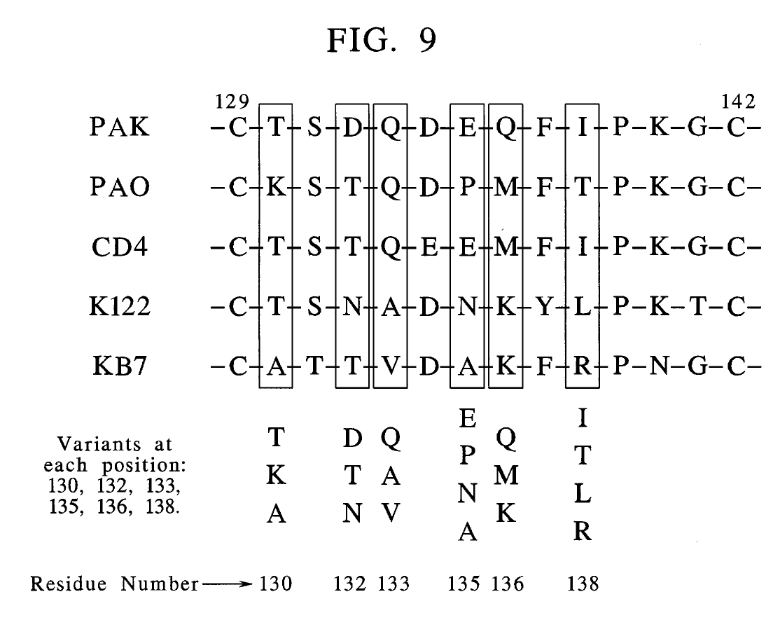

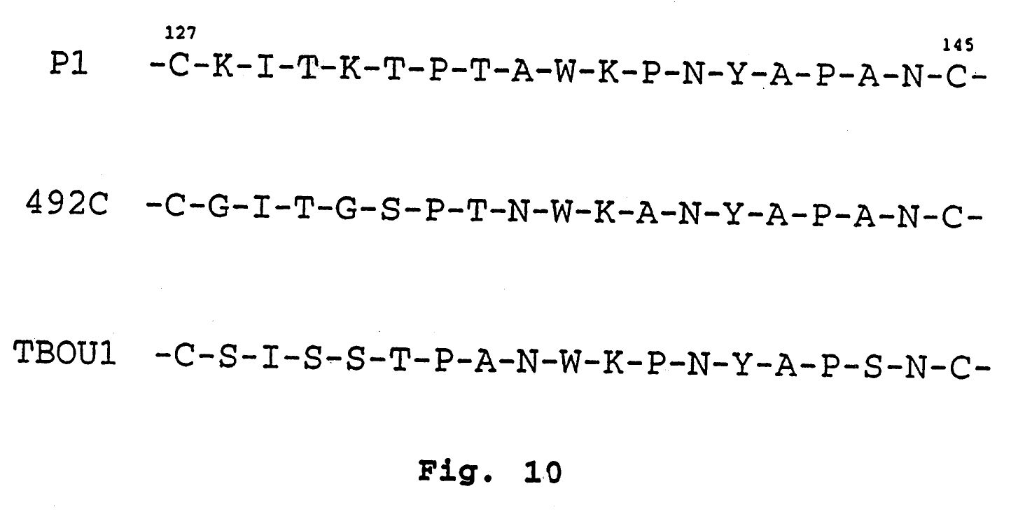

FIG. 9 describes five sequences of the C-terminal peptides from Pseudomonas aeruginosa. These sequences from the protein Pilin encompass the binding domain responsible for attachment to the host cell surface receptors. The peptides are 14-residues in length and contain disulfide-bridges between the cysteine residues at positions 129 and 142. The boxed regions define homologous positions in the 5 sequences which are the most variable in amino acid composition and contain 3 to 4 different amino acid variants across the 5 strains (see FIG. 9 positions 130, 132, 133, 135, 136 and 138). Other positions in these sequences contain the same amino acid in a position (i.e. position 139 contains proline) or have only 1 amino acid different across the 5 strains (i.e. position 137 contains the residues phenylalanine and tyrosine). The peptide library containing all possible variants of the PAK sequence in the boxed positions would contain 1296 (3×3×3×4×3×4) peptides including the native PAK sequence itself, excluding any residues in these 6 positions that are not found in the five strains. Two of these sequences, the single mutant E135A and the multiple mutants CS1, CS2, CS3, and CS4 provide cross-protection against various Pseudomonas aeruginosa strains challenged in the mouse model (FIGS. 2-7). The emphasis has been on residues between the positions 129 and 142 because, to date, all epitopes that have been mapped, in both polyclonal and monoclonal antibodies raised to larger C-terminal fragments containing this region, show that the epitopes lie within the loop structure. FIG. 10 contains the sequences of 3 other native PA C-terminal sequences (P1, SEQ ID NO: 12, 492C, SEQ ID NO: 13, and TBOU1 (SEQ ID NO: 14) that contain larger loops than the 5 shown in FIG. 9. Complete protection will only be achieved if mutant sequence(s) should cross-react with these sequences.

Detailed Studies

Previously, native polyclonal antisera (17-O1), raised against the native PAK C-terminal pilin synthetic sequence containing the intrachain disulfide bridge (AcPAK (128-144)OH) was shown to be cross-reactive with synthetic peptide (AcPAO (128-144)OH) and pilin from strain PAO, a result confirmed herein. See, Lee, K. K., et al., Mol. Microbiol. 3:1493-1499 (1989); Lee, K. K., Infect. Immun. 57:520-526 (1989); and Lee, K. K., et al., Infect. Immun. 58:2727-2732 (1990). Epitope mapping studies using single alanine substitution analogs of the PAK sequence were performed to determine the side-chain specificity of antisera raised to the reduced and disulfide-bridged (oxidized) immunogens.

The relative importance of each of the residues in the epitopic region is based on the analysis of the apparent binding constants (Ka). The Ka's at each position for both the native sequence (K_{N}) and the single alanine substitution analogue (K_{S}) are displayed as the ratio K_{N}/K_{S} (Table 1).

The side-chains are divided into three types based on the K_{N}/K_{S} ratios: critical, important and nonessential to antibody binding. A critical side-chain is one in which substitution by alanine decreases binding affinity more than 1,000-fold as compared to the native sequence. On the other hand, if the decrease in binding affinity is less than 10-fold, the side-chain is considered as nonessential. Side-chains whose contribution falls between these two extremes are defined as important. In antiserum 17-R1 the analysis reveals that the residues in positions 132, 134 to 136, 138 and 139 of oxidized AcPAK(128-144)OH are important to binding this native PAK peptide structure. Residue F137 is particularly critical for antibody binding as shown by the more than 10,000-fold decrease in binding affinity which occurs when phenylalanine is substituted by alanine in the native PAK sequence (Table 1; column, 17-R1; row, F137A). Antiserum 17-O1, raised to oxidized AcPAK(128-144)OH, has three residues at positions 134, 136 and 137 which are all classified as critical to antibody binding to native oxidized PAK peptide (Boxed residues; Table 1). In fact, antiserum raised to the analogue F137A, conjugated to Keyhole Limpet Haemocyanin, fails to bind native PAK pili indicating the importance of this side-chain, in the peptide immunogen, for maintaining recognition of the native pilin protein. See, Wong, W. Y., Ph.D. Thesis: “Synthetic Peptide Approaches to Study the Adherence Binding Domain of the Pilin Protein of Pseudomonas aeruginosa Strain PAK,” University of Alberta, Edmonton, Alberta, Canada, p. 222 (1994); and Wong, W. Y., et al, “Pseudomonas Pilin Vaccine,” in The 8th Annual North American Cystic Fibrosis Conference, Orlando, Fla. (1994). Previous NMR studies on the conformation of the native immunogen showed that F137 is buried in the hydrophobic core of the folded peptide.

The position of F137 is critical for maintaining conformation of the immunogen and therefore critical to generating antiserum that recognizes the native sequence. See, McInnes, C., et al., Biochemistry 32:13432-40 (1993). Furthermore, D134A and Q136A peptides bind to the two antisera 17-R1 and 17-O1 with dramatically different affinities (D134A binds 10-fold weaker to antiserum 17-R1 but 1600-fold weaker to antiserum 17-O1 and Q136A binds 390-fold weaker to antiserum 17-R1 compared to 3200-fold weaker to antiserum 17-O1 relative to the native PAK peptides). While positions 133 and 139 have maintained an important role in antibody binding (10<K_{N}/K_{S}<1000) in antiserum 17-O1, positions 132, 135 and 138 have become unimportant to antibody binding (K_{N}/K_{S}<10). This suggests that the oxidation of the peptide (formation of the disulfide bridge) in the immunogen results in a change in conformation that redefines residues critical to antibody binding to the native PAK peptide sequence. In other words, the disulfide bridge in the immunogen changes the side-chain specificity and enhances cross-reactivity of the resulting antisera (Table 1, K_{PAK}/K_{PAO} ratios of antisera 17-R1 and 17-O1 are 4,400 vs 1,270, respectively).

Ideally, it is desired to have the cross-reactivity ratio between PAK and any other strain to approach unity. As a starting point, peptide antigens were constructed by substituting out side-chains important for strain-specificity but unimportant for cross-reactivity. For example, peptide E135A binds 480-fold weaker to strain-specific antiserum 17-R1 than the native peptide while E135A binds only 2-fold weaker to the more cross-reactive antiserum 17-O1. Thus, E135 seems important for strain-specificity but as the antiserum becomes more cross-reactive E135 becomes unimportant.

Initially, two peptide immunogens with single alanine substitutions at positions 135 and 138 were prepared. In addition, in order to specifically enhance cross-reactivity to strain PAO, a proline analogue at position 135 of the PAK sequence was synthesized. Proline was chosen because it is found in the corresponding position in the PAO native sequence. Since position E135 in the native PAK sequence was non-essential for binding to antiserum 17-O1, substitution of this residue by proline should enhance cross-reactivity of the antiserum prepared against the immunogen E135P.

The epitopes recognized by these antibodies prepared to the disulfide bridged peptides E135A, E135P and I138A were mapped by competitive ELISA assays using AcPAK(128-144)OH single alanine replacement analogs (Table 1; column, PEPTIDE) as competitive inhibitors in the presence of PAK native peptide on plates coated with PAK pili. The results of the substitution analyses show that the epitope (Table 1) recognized by each of these antisera spans the residues 132 to 140 in the native sequence AcPAK(128-144)OH similar to antisera 17-R1 and 17-O1 previously reported. See, Wong, W. Y., et al., “Pseudomonas Pilin Vaccine,” in The 8th Annual North American Cystic Fibrosis Conference, Orlando, Fla. (1994). Analysis of the K_{N}/Kₛ ratios, for each of these antisera, indicates that the importance of the individual residues has changed when compared to antiserum 17-O1. For example, dramatic changes occur in antisera I138A and E135P. In antiserum I138A the most critical residue for antigen binding is Q133. The previously critical residue in antiserum 17-O1, F137, is now unimportant to antigen binding. In antiserum E135P the residues critical for antigen binding are Q136 and K140. Similarly, the importance of F137 is dramatically reduced compared to antiserum 17-O1. Examination of the results for antiserum E135A reveals that deletion of the glutamic acid side-chain at position 135 and its replacement by the methyl group of alanine has not affected the antibodies affinity for this analogue (K_{N}/K_{S}=1) in comparison to the native PAK sequence. Therefore E135 is considered unimportant to antibody affinity. Furthermore, the analysis of cross-reactivity shows that the antiserum E135A exhibits similar cross-reaction with native PAO pilin sequence (Table 1; compare K_{PAK}/K_{PAO} ratios for 17-O1 and E135A; 1,270 and 1,350, respectively).

Interestingly, the antiserum E135A has an increased affinity (˜3-fold) for both PAK and PAO native peptide sequences (Table 2; row, E135A; columns, PAK and PAO; +3.2 and +3.0, respectively). As for other side-chain effects in the epitopic region, changes have occurred most noticeably in the K_{N}/K_{S} values observed for positions Q133 (Table 1; column, E135A; row, Q133A). In antiserum E135A, the K_{N}/K_{S} value has increased by a factor of 40-fold to a value of 830 in comparison to the value of 20 found in antiserum 17-O1.

The effects on residues Q136 and F137 are less pronounced yet remain large. The K_{N}/K_{S} value for Q136 is reduced by a factor of approximately 6-fold while the value associated with F137 indicates a change however the precise magnitude is undetermined (Table 1; compare columns, 17-O1 and E135A; rows, Q136A; K_{N}/K_{S}=3,200 and 520 and F137A; K_{N}/K_{S}=>10,000 and >4,800, respectively). Q136 and F137 remain important and critical to binding. Analysis of residue D134 shows that the result of substitution of alanine at position 135 has had little effect on the critical nature of this side-chain (Table 1; compare columns, 17-O1 and E135A; row, D134A; K_{N}/K_{S}=1,600 and 1,200, respectively).

Finally, the result of the alanine substitution at position 135 has generally increased the importance of residues Q133, 1138 and P139 to antiserum binding. The overall assessment of this substitution suggests that it has caused changes throughout the epitope which have decreased the contributions of some of the critical residues, namely Q136, but raised the contribution to antiserum binding of less critical but important residues, Q133, 1138 and P139.

In summary, the results show that the epitopic region is similar in all the antisera 17-R1, 17-O1, E135A, I138A and E135P but side-chain specificities vary. These results demonstrate that it is possible to manipulate epitopic sequences by single amino acid mutation in the immunogen and retain binding affinity to the native antigen of the same order of magnitude as that of the native antiserum (Table 2; column PAK) and that the side-chain specificities observed are different in each of the antisera tested (Table 1).

Comparison of the Cross-Reactivity of the Different Antisera

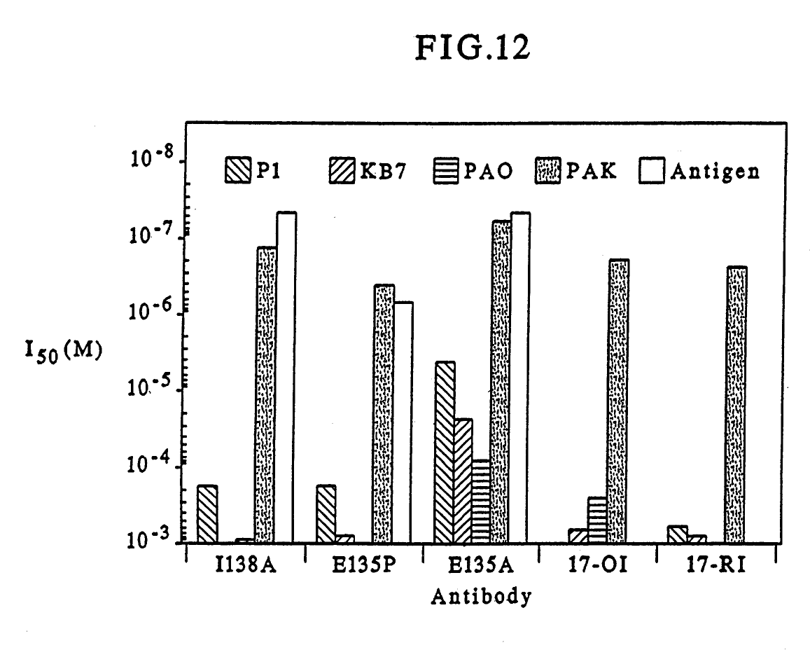

In this analysis, the cross-reactivity of five antisera was studied on microtiter plates coated with native PAK strain pili (Table 2). Competition assays were performed using synthetic peptides specifically representing the homologous C-terminal regions of each of four Pseudomonas aeruginosa strains AcPAK(128-144)OH, AcPAO(128-144)OH, AcKB7(128-144)OH and AcP1(126-148)OH) or the corresponding analogs of AcPAK(128-144)OH. By plotting the 150 values of the four strain specific synthetic peptides (strains PAK, PAO, KB7 and P1) and the analogs in separate competition assays with the antisera (FIG. 12 and Table 2), the cross-reactivity of the antisera to the different strains is evaluated. Assays included the native PAK pilin sequence AcPAK(128-144)OH as a control. Antisera E135P, I138A and E135A were all generated to synthetic peptide antigens.

Examination of the results from antisera 17-R1 and 17-O1 indicates that formation of the disulfide bridge between residues 129 and 142 decreases the K_{PAK}/K_{PAO} ratio and therefore is important for cross-reactivity to PAO strain (Table 1; K_{PAK}/K_{PAO}=4,400 and 1,270, respectively). Examination of the I₅₀ values of 17-O1 and 17-R1 (Table 2) shows that these two antisera have similar affinities for PAK (0.19 and 0.23 10⁻⁶ M, respectively) while the affinity of antiserum 17-O1 is 4-fold higher for PAO (241 and 1,014 10⁻⁶ M, respectively).

Cross-reactivity to PAO was also examined in the other antisera: E135A, I138A and E135P. The results shown in Tables 1 and 2 demonstrate that antiserum E135A is cross-reactive to PAK and PAO and the magnitude of this cross-reactivity is equivalent to that of 17-O1 (K_{PAK}/K_{PAO}=1,350). However, the I₅₀ values (FIG. 12 and Table 2) of both peptides, PAK and PAO, for antiserum E135A increased by a factor of 3-fold over that of antiserum 17-O1 (I₅₀; PAK, 0.06 vs. 0.19 10⁻⁶ M; PAO, 81 vs. 241 10⁻⁶ M, respectively). Antiserum I138A was also analyzed for cross-reactivity to PAK and PAO. The affinity for PAK remained similar to that of 17-O1 (Table 2, I₅₀; 0.13 vs. 0.19 10⁻⁶ M for I138A and 17-O1, respectively) while that of PAO is reduced about 4-fold (Table 2, I₅₀; 909 vs. 241 10⁻⁶ M for I138A and 17-O1, respectively). Therefore, antiserum I138A is about 6-fold less cross-reactive than 17-O1 (Table 1; K_{PAK}/K_{PAO}=7000 vs. 1,270).

A proline residue occupies position 135 of the native PAO sequence (FIG. 1). Since position 135 in the native PAK sequence was not critical to PAK/PAO cross-reactivity (Table 1; columns 17-O1 and E135A; row K_{PAK}/K_{PAO}=1,270 vs. 1,350, respectively) we reasoned that an immunogen containing an E135P substitution in the native PAK sequence (AcPAK(128-144)OH) might generate antiserum with an enhanced cross-reactivity to PAO. Peptide E135P was synthesized, antiserum was generated in rabbits and cross-reactivity to PAK and PAO was evaluated as before. The results show (Table 2 and FIG. 12) that the affinity for PAK has decreased by about a factor of 2 (0.41 vs. 0.19 10⁻⁶ M for E135P and 17-O1, respectively) and the affinity for PAO has decreased approximately 20-fold compared to antiserum 17-O1 (4,751 vs. 241 10⁻⁶ M for E135P and 17-O1, respectively). These results show that the proline substitution at position 135 in the native PAK sequence has significantly decreased the affinity of the homologous antiserum (E 135P) for the native PAO sequence which in turn has greatly decreased the antiserum's cross-reactivity (Table 1, K_{PAK}/K_{PAO}=11,600 vs. 1,270) rather than increased it. In effect, the substitution of proline in the native PAK sequence at position 135 has resulted in the production of antiserum which is highly specific for native PAK peptide in the presence of native PAO peptide while the affinity for native PAK peptide has been lowered by approximately 2-fold (Table 2; row E135P; columns PAK and PAO).

The cross-reactivities to native peptide sequences KB7 and P1 were also studied in all the antisera. A summary of the results is found in FIG. 12 and Table 2. These data show that, although 17-O1 does exhibit cross-reactivity to native PAO sequence, the antisera 17-R1 and 17-O1 do not exhibit broad cross-reactivity as judged by their affinities for the other heterologous peptides from strains P1 and KB7. In addition, antisera I138A, E135A and E135P were examined for their affinity for their respective immunogens (Table 2; column, Antigen I₅₀). In each case the antisera demonstrate high affinity and cross-reactivity for both the native PAK sequence and the analogs used to generate the antisera (Table 2 and FIG. 12). As for cross-reactivity to the heterologous strains, PAO, P1 and KB7, only antiserum E135A exhibits broad cross-reactivity with I₅₀ values in the range 10-5M to 10-8M (0.06, 81, 22 and 4 10⁻⁶ M for PAK, PAO, KB7 and P1, respectively, FIG. 12 and Table 2). Not only is the antiserum cross-reactive, the cross-reactivity is significantly enhanced to peptides from strains KB7 and P1 in comparison to the native antiserum 17-O1 and to all the other antisera tested. For example, the affinity of antiserum E135A for peptides PAK and PAO has increased 3-fold and for peptides KB7 and P1, 32 and 300-fold, respectively, compared to antiserum 17-O1. In addition, the data in Table 2 also demonstrates, the affinity of antiserum E135A is from 2- to 7-fold higher than the other antisera for PAK peptide (ratios of I₅₀ values), 3- to 59-fold higher for PAO peptide, 32- to 60-fold higher for KB7 peptide and 44- to 300-fold higher in affinity for PI peptide. In FIG. 12, this is reflected in the histogram of I₅₀ values (log 10 of the molar concentration of peptide required to achieve 50% inhibition of antiserum E135A binding to PAK pilin) reported for E135A antiserum.

As for the antisera I138A and E135P, the data shows that they are not able to effectively bind heterologous peptides PAO and KB7. However, all three antisera, I138A, E135P and E135A, demonstrate affinity for native peptide sequence P1, which is 7- to 300-fold better than antiserum 17-O1 (176 10⁻⁶ M for I138A and E135P and 4 10⁻⁶ M for E135A compared to 1,217 10⁻⁶ M for 17-O1, FIG. 12 and Table 2). This is surprising since peptide sequence P1 is very different from the sequences of PAK, PAO and KB7 (FIG. 1) in length, amino acid composition and size of the disulfide loop.

In order to test the efficacy of E135A, this peptide was conjugated to tetanus toxoid and the conjugate was used in our A.BY/SnJ mouse model to test active immunization. The A.BY/SnJ mice are less resistant to Pseudomonas aeruginosa infection than normal laboratory mice. It is therefore unnecessary to use immunosuppressive procedures to demonstrate antibody-induced protection. Since antisera E135A was least cross-reactive to PAO strain (I₅₀, 0.06 10⁻⁶ M for PAK vs. 81 10⁻⁶ M for PAO; K_{PAK}/K_{PAO}=1,350) compared to the other strains (Table 2), it was important to demonstrate the level of cross-reactivity could still offer protection to challenge from strain PAO FIG. 2 demonstrates that challenge with 2×10⁶ cfu (˜3 LD₅₀; PAO strain) results in protection against lethal challenge. As the plot of survivors vs. hours after challenge shows, the two groups of mice (10 mice per group) that were immunized with E135A conjugate or PAO conjugate demonstrated increased survival times when compared with those groups which had been immunized with PAK conjugate or the adjuvant alone as a control. This result demonstrated the enhanced efficacy of this peptide, E135A, over the corresponding PAK native sequence. The result is even more important since examination of the two sequences, PAO and E135A, shows that these sequences differ at 7 positions. The results demonstrate that the replacement of E135 by alanine has created an immunogen, which is, for all but one residue, identical to the PAK native sequence—a sequence which does not provide protection against PAO challenge—yet this new sequence is providing protection similar to that of the native PAO sequence.

Mapping studies were used to define specific residues responsible for peptide binding to the antisera. The cross-reactive native polyclonal antisera described here, 17-O1, binds to both PAK and PAO native peptides. Examination of the mapping results for antiserum 17-O1 (Table 1; D134, Q136 and F137; K_{N}/K_{S} values) reveals that D134, Q136, and F137 are all critical residues for binding to the native protein.

Antisera was raised to a peptide analog in which E135 was replaced by an alanine at position 135 in native PAK sequence, AcPAK(128-144)OH. This substitution resulted in a large decrease in the contribution to binding from Q136 (6-fold). The contributions from F137 and D134 side chains have remained large while contributions from residues Q133, I138, and P139 were all significantly enhanced (6- to 450-fold) by this substitution. Cross-reactivity of antiserum E135A to PAO remained the same as with antiserum 17-O1.

The antisera I138A and E135P show some of the most dramatic effects on substitution. In antiserum I138A, the loss of the hydrophobic contribution at I138, by substitution with alanine, has substantially increased the importance of Q133 (K_{N}/K_{S}=250,000) but cross-reactivity to PAO sequence decreases by a factor of 6-fold over that of antiserum 17-O1. Antiserum E135P demonstrates effects in both the contributing residues and cross-reactivity. This antiserum was generated by substitution of E135, in the PAK native sequence, by the proline residue from position 135 in the PAO native sequence (FIG. 1). The rationale behind this substitution was based on the hypothesis that a more cross-reactive antibody response is generated by using a peptide immunogen in which a side-chain unimportant for PAK strain specificity could be replaced by a side-chain from a different strain that would enhance cross-reactivity to that strain. The mapping results for antiserum E135P show that the critical residues for protein recognition are Q136 and K140 with contributions from D134 and I138. This is a significant shift from the results obtained using antisera 17-O1. With respect to cross-reactivity, antiserum E135P is very specific for strain PAK (Table 1; K_{PAK}/K_{PAO}=1,600). The value of I₅₀ for PAO (FIG. 12 and Table 2; column PAO; 4,751 10⁻⁶ M) indicates that native PAO peptide binds very weakly while PAK peptide binding is similar to that of antiserum 17-O1 (FIG. 12 and Table 2; column PAK; 0.41 10⁻⁶ M). An even more specific example of a side-chain effect on contributions to binding can be demonstrated with antisera, which were raised in response to single alanine substitution analogs Q136A and F137A. These antisera failed to bind to the native pili indicating that these two residues are essential for immunogenicity and recognition of the native pilin epitope sequence. See, Wong, W. Y., Ph.D. Thesis: “Synthetic Peptide Approaches to Study the Adherence Binding Domain of the Pilin Protein of Pseudomonas aeruginosa Strain PAK,” University of Alberta, Edmonton, Alberta, Canada, p. 222 (1994); and Wong, W. Y., et al., “Pseudomonas Pilin Vaccine,” in The 8th Annual North American Cystic Fibrosis Conference, Orlando, Fla. (1994). Based on the evidence cited here it would be reasonable to suggest that there are both peptide backbone structural features and amino acid side-chain characteristics which, in combination, determine immunogenicity and binding to antisera.

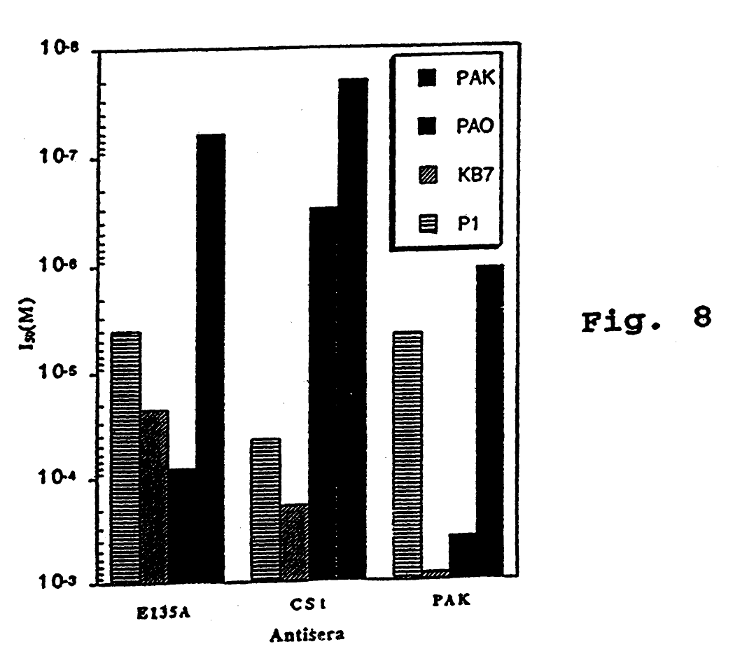

Similar studies were carried out using antisera against the E135A, CS1, and PAK peptide in reaction with PAK, PAO, KB7, and P1, as indicated in FIG. 8. In addition to the results previously reported for cross-reactivity with the E135A antigen, the CS1 antigen is seen to give very high cross-reactivities with PAK and PAO, and moderate cross-reactivities with KB7 and P1. As seen from the data in FIGS. 4 and 6, CS1 was effective in protecting the subject animal against both P1 and KB7 infection.

Two-dimensional 1H NMR spectroscopy was used to determine the antigenic determinants recognized by the Pseudomonas aeruginosa cross-reactive monoclonal antibody PAK-13. The results demonstrated that residues for which spectral changes were observed upon antibody binding were different for each of the peptides. However, these residues are confined to common structural features that comprise each peptide antigen, namely, two β-turns and a hydrophobic pocket.

The examples illustrate the invention, but are in no way intended to limit it. Unless otherwise stated all reagents were reagent grade. Bovine serum albumin was purchased from SIGMA CHEMICAL CO., St Louis, Mo. Goat anti-mouse IgG horseradish peroxidase conjugate was purchased from Jackson IMMUNORESEARCH LABORATORIES, INC., West Grove, Pa. 2,2′-azino-di-(3-ethylbenzthiazoline sulfonic acid) was purchased from BOEHRINGER MANNHEIM, LAVAL, PQ. Untreated polystyrene 96-well flat bottom microtiter plates were purchased from COSTAR CORP., Cambridge, Mass. FREUND'S complete and incomplete adjuvants were purchased from GIBCO LABORATORIES, LIFE TECHNOLOGIES INC., Grand Island, N.Y. Keyhole limpet haemocyanin was purchased from SIGMA CHEMICAL COMPANY, St. Louis, Mo. and tetanus toxoid was purchased from PASTEUR MERIEUX CONNAUGHT LABORATORIES, North York, ON, Canada. Adjuvax adjuvant was purchased from ALPHA-BETA TECHNOLOGY, One Innovation Drive, Worcester, Mass.

### EXAMPLE 1

Bacterial pili

The bacterial pili employed in this study were obtained from the Pseudomonas aeruginosa strain PAK/2pfs. Purification of the pili was as previously described. See, Paranchych, W., et al., Can. J. Microbiol. 25:1175-1181 (1979).

The synthetic pilin peptides and their analogs were prepared following the general procedure for solid-phase peptide synthesis (SPPS) as described by Erickson and Merrifield (See, Erickson, B. W. and Merrifield, R. B., “Solid-phase peptide synthesis”, in THE PROTEINS, (Neurath, H. and R. L. Hill, Eds.), pp 255-527, Academic Press, New York (1976)) and the SPPS protocols, purification, and characterization of the peptides have been described (See, Wong, W. Y., et al., Protein Sci. 1:1308-18 (1992)).

The preparation of peptide conjugates has been described by Lee et al. See, Lee, K. K., et al., Mol. Microbiol. 3:1493-1499 (1989). Peptides containing the photo reactive group, benzoyl benzoic acid, attached to the N-terminal end were conjugated to the protein carrier, keyhole limpet haemocyanin (KLH). The peptides (2-5 mg) dissolved in 10-20 μl of water were mixed with 500 μl of 8M urea containing KLH (10 mg). This solution was then irradiated at 350 nm for one hour at 48° C. in a RPR 208 preparative reactor (RAYONET, THE SOUTHERN NEW ENGLAND ULTRAVIOLET CO., Middletown, Conn.) equipped with RPR 350 nm lamps. Unconjugated peptides were removed by successive dialysis against 8M urea, 1M urea, PBS at pH 7.2. The product was lyophilized and the peptide incorporation determined by amino acid analysis.

Peptides containing the photo reactive group, benzoyl benzoic acid, attached to the N-terminal end were conjugated to the protein carrier, tetanus toxoid (TT). The peptides (2-5 mg) dissolved in 10-20 μl of water were mixed with 500 μl PBS at pH 7.2 containing TT (10 mg). This solution was then irradiated at 350 nm for one hour at 48° C. in a RPR 208 preparative reactor (RAYONET, THE SOUTHERN NEW ENGLAND ULTRAVIOLET CO., Middletown, Conn.) equipped with RPR 350 nm lamps. Unconjugated peptides were removed by dialysis against PBS at pH 7.2. The product was lyophilized and the peptide incorporation determined by amino acid analysis.

### EXAMPLE 2

Antipeptide Antisera

A group of 3 New Zealand White rabbits were used for immunization in a peptide conjugate experiment. The rabbits were used at eight weeks of age and at approximately 2 kilograms in weight. Prior to immunization, a small sample of blood (5 ml) was drawn from the vein of the rabbit's ear and used as preimmune sera. The peptide-KLH conjugates were dissolved in sterile PBS and mixed with equal volume of Freund's complete adjuvant. This mixture was then thoroughly mixed until a thick white emulsion formed. A two-site (subcutaneous and intramuscular) injection with 200 μl/site of the emulsion was performed on each rabbit. The amount of peptide-conjugate injected was 200 to 350 μg/rabbit, depending on the degree of incorporation of the peptide analog on KLH. Two booster injections were administered at two week intervals using the same amount of the peptide conjugate emulsified with Freund's incomplete adjuvant. Blood samples (5 ml each) were taken 10 days after the third injection. The antiserum titer was then determined by direct ELISA. Further booster injections and sera collections were subsequently performed at 4 week intervals.

### EXAMPLE 3

Active Immunization with Peptide Conjugate

A.BY/SnJ mice were actively immunized at week 0 with 40-50 μg of peptide-tetanus toxoid conjugate mixed with 100 μg of Adjuvax as adjuvant in 50 μl of 10 mM PBS at pH 7.2. Control mice were given 50 μ of 10 mM PBS at pH 7.2 containing 100 μg of Adjuvax. The mice were then boosted at weeks 2 and 4 and challenged with 2×10⁵ cfu of Pseudomonas aeruginosa strain PAO (˜3×LD₅₀) at week 6. Each test group and the control group consisted of ten animals. The effectiveness of the vaccine was determined by the survival rate of the test animals up to 48 hours.

### EXAMPLE 4

Enzyme-Linked Immunosorbent Assay (ELISA)

Competitive ELISAs were performed according to the following protocol. Untreated 96-well flat bottom microtiter plates were coated with 0.2 μg/well of PAK pili for 1 hour at 37° C. and blocked with 5% (wt/vol) BSA (100 μl/well) dissolved in 10 mM PBS, pH 7.4, containing 150 mM sodium chloride for 8 hours at 4° C. The plates were then washed, five times, with 10 mM PBS, pH 7.4, containing 150 mM sodium chloride and 0.05 % (w/v) BSA (buffer A). Raw sera containing polyclonal antibodies (dilution, 1:5,000) were preincubated with equal volumes of serially diluted epitopic peptide pilin sequences for 1 hour at 37° C. These solutions were added (100 μl/well) to the pili coated wells on the microtiter plate. Following incubation for 2 hours at 37° C., the plates were washed, five times, with buffer A. A goat anti-rabbit IgG horseradish peroxidase conjugate, that had been diluted 1:5000 with buffer A, was added to the wells (100 μl/well). A second incubation was performed for 2 hours at 37° C. and the plates were washed, five times, with buffer A 2,2′-azino-di-(3-ethylbenzthiazoline sulfonic acid) (ABTS) (1 mM) in 10 mM sodium citrate buffer, pH 4.2, containing 0.03% (v/v) hydrogen peroxide was used for detection. Finally, the absorbance at 405 nm was determined by using a TITERTEK MULTISKAN PLUS MK II microplate reader (FLOW LAB INC., Mclean, Va.).

The percentage inhibition (%Inhibition) for each competitive assay was calculated by the following formula:

% Inhibition=100% −[(A405 Competition/A405 No Competition)*100%]

The percent inhibition at each peptide concentration was determined from the mean value of 8 repetitions. The competitive binding profile was plotted as %Inhibition (±SD) vs Log10 (competitor concentration). The I₅₀ value (competitor concentration that causes 50% inhibition) was determined by using the software KALEIDAGRAPH (SYNERGY SOFTWARE, Reading, Pa.). The apparent association constant (Ka) of the antiserum for each peptide analogue can be calculated by the formula Ka=(I₅₀)−1 as described by Nieto et al. See, Nieto, A., et al., Mol. Immunol. 21:537-543 (1984).

TABLE 1
Epitope Mapping of Five Anti-PAR Peptide Antibodies

| | | | K_{N}/K_{S}^{a} Ratios | | |
|---|---|---|---|---|---|
| | | | of the Antipeptide Antibodies | | |
| Peptide^{b} | 17-R1 | 17-01 | E135A | I138A | E135P |
| K128A | 1 | 1 | <1 | — | — |
| C129 | — | — | — | — | — |
| T130A | 2 | 1 | <1 | — | — |
| S131A | 2 | 1 | <1 | — | — |
| D132A | 10 | 6 | 1 | <1 | <1 |
| Q133A | 9 | 20 | 830 | >250,000 | <1 |
| D134A | 10 | 1,600 | 1,200 | 71 | 267 |
| E135A | 480 | 2 | 1 | 3 | 3 |
| Q136A | 390 | 3,200 | 520 | 60 | >10,000 |
| F137A | >10,000 | >10,000 | >4,800 | <1 | 20 |
| I138A | 18 | 1 | 450 | <1 | 208 |
| P139A | 280 | 14 | 390 | <1 | 1 |
| K140A | 2 | 2 | 8 | 120 | >100,000 |
| G141A | 1 | 1 | 1 | — | — |
| C142 | — | — | — | — | — |
| S143A | 1 | <1 | 1 | — | — |
| K144A | 1 | 1 | 1 | — | — |
| K_{PAK}/K_{PAO}^{c} | 4,400 | 1,270 | 1,350 | 7,000 | 11,600 |

^{a}The values of the apparent binding constants (Ka) are expressed as K_{N} for native peptide PAR and K_{S} for the corresponding analogue being tested. Values for K_{N}/K_{S} ratios that are >1,000 are boxed and the side-chain at this position is considered critical for antibody binding.
^{b}PEPTIDE designates the native sequence of PAK pilin peptide and the residue within this sequence which has been substituted by alanine. The cysteines (129 and 142) are retained for the purposes of disulfide bridge formation. There are 15 single alanine substitution analogues each designated by the position of substitution. For example,
#K12SA represents position Lys 128 substituted by Ala.
^{c}Cross-reactivity of an antiserum is expressed as the ratio K_{PAK}/K_{PAO}. A value of 1 would indicate that the antiserum has the same affinity for native PAO peptide sequence as it does for native PAK peptide sequence in competition ELISA assay and therefore has excellent cross-reactivity. The larger the value of
#K_{PAK}/K_{PAO} the more specific the antiserum is for PAK native sequence and the less cross-reactive it is with the native PAO sequence.

TABLE 2
Antisera Affinity Results

| | | Peptide Competitor^{c} | | | | | | | |
|---|---|---|---|---|---|---|---|---|---|
| | | PAK | | PAO | | KB7 | | PI | |
| Anti- | Antigen^{b} | I₅₀^{d} | | I₅₀ | | I₅₀ | | I₅₀ | |
| serum^{a} | I₅₀ (μM) | (μM) | A^{e} | (μM) | A | (μM) | A | (μM) | A |
| I138A | 0.05 | 0.13 | +1.5 | 909 | −3.8 | 1,320 | −1.9 | 176 | +6.9 |
| E135P | 0.70 | 0.41 | −2.2 | 4,751 | −19.7 | 835 | −1.2 | 176 | +6.9 |
| E135A | 0.05 | 0.06 | +3.2 | 81 | +3.0 | 22 | +32.0 | 4 | +304 |
| 17-01 | 0.19 | 0.19 | 1.0 | 241 | 1.0 | 704 | 1.0 | 1,217 | 1.0 |
| 17-R1 | — | 0.23 | −1.2 | 1,1014 | −4.2 | 845 | −1.2 | 608 | +2.0 |

^{a}Antisera raised to the native PAK peptide sequence 128-114 in the disulfide-bridged (oxidized) form (17-01) and the reduced form (17-R1) and the single substitution analogues of this disulfide-bridged native PAK sequence (I138A, I135P and I135A).
^{b}Antigen I₅₀ represents the I₅₀ value of the corresponding disulfide-bridged peptide analogue that was used to raise antiserum I138A, I135P and I135A or the disulfide-bridged native PAK sequence and reduced native PAK sequence used to raise antiserum 17-01 and 17-R1, respectively.
^{c}Peptide competitors in the ELISA assays were the oxidized (disulfide-bridged) peptides of the receptor binding domains of pilin strains PAK, PAO, KB7 and P1 (FIG. 1).
^{d}The I₅₀ values indicate the concentration of peptide required to produced 50% inhibition of antibody binding to PAK pilin.
^{e}“A” represents an increase or decrease in the affinity of the antiserum against a particular native sequence (PAK, PAO, KB7 and P1) compared to the affinity of the antiserum of native PAK (17-01). A positive value is the number fold improvement in affinity whereas a negative value represents the number fold decrease in affinity
#of the antiserum for that particular native sequence compared to native PAK antiserum 17-01.

### EXAMPLE 5

Immunization to Elicit an Antibody Response to the Consensus Sequence Peptide of P. aeruginosa

BALB/c mice were immunized by intraperitoneal injection (i.p.) on days 0, 14, and 43 with the consensus sequence peptide tetanus toxoid conjugate (CS1-TT) (˜4 μg CS1 peptide+16 μg tetanus toxoid) +/−2% Alhydrogel (alum) (SUPERFOS BIOSECTOR, Denmark, Cat. # SE2000-1). These conjugates were administered using a sterile 0.5 mL tuberculin syringe with a 27-gauge needle (BECTON DICKINSON, Cat. #305620). The mice were bled at day 21 and 51 and direct ELISAs were done to determine CS1 titers.

TABLE 3a
Immunization Protocols

| Day | Procedure | Procedure |
|---|---|---|
| 0 | Immunize i.p. + | Immunize i.p. + alum |
| | alum | |
| 14 | Immunize i.p. | Immunize i.p + alum |
| 21 | Bleed | Bleed |
| 43 | Immunize i.p. | Immunize i.p. |
| 51 | Bleed | Bleed |

TABLE 3b
Alternate Immunization Protocols Used to Generate Antibodies

| Day | Day | Day | Day | Day | Procedure |
|---|---|---|---|---|---|
| 0 | 0 | 0 | 0 | 0 | Immunize i.p. + alum |
| 7 | 7 | 7 | 10 | 7 | Immunize i.p. + alum |
| 28 | 28 | 28 | 28 | 28 | Immunize i.p. + alum |
| 42 | 42 | 42 | | 42 | Immunize i.p. + alum |
| 57 | 57 | 57 | | 57 | Immunize i.p. + alum |
| 71 | 74 | 74 | | | Immunize i.p. + alum |
| | 88 | | | | |
| pre-fusion | pre-fusion | pre-fusion | pre-fusion | Pre-fusion | Boost i.p. and i.v. 4 |
| boost | boost | boost | boost | boost | days pre-fusion |

As shown in Tables 3a and 3b, genetically modified mice have been immunized by intraperitoneal injection (i.p.) at various timepoints with the consensus sequence peptide tetanus toxoid conjugate (CS1-TT) at varying levels of antigen (30-100 μg) with or without 2% Alhydrogel (alum) (SUPERFOS BIOSECTOR, Denmark, Cat. # SE2000-1). These conjugates were administered using a sterile 0.5 mL tuberculin syringe with a 27-gauge needle (BECTON DICKINSON, Cat. #305620). The mice were bled at varying timepoints and direct ELISA was done to determine CS1 titers.

TABLE 4
Example of Mouse Polyclonal Antibody Titer Elicited by the
Consensus Sequence Immunogen (Pre-fusion Bleed)

| Anti-Sera Dilution | O.D. |
|---|---|
| 1:1,000 | 3.794 |
| 1:5,000 | 3.794 |
| 1:25,000 | 3.794 |
| 1:125,000 | 3.794 |
| 1:625,000 | 2.724 |
| 1:3,125,000 | 0.757 |
| 1:18,625,000 | 0.141 |

TABLE 5
Example of Mouse Sera Isotypes Elicited by Consensus
Sequence Immunogen

| Isotype | O.D. |
|---|---|
| IgM | 0.96 |
| IgG1 | 3.79 |
| IgG2a | 2.92 |
| IgG2b | 2.85 |
| IgG3 | 0.73 |

### EXAMPLE 6

Fusion Protocol for Generating Monoclonal Antibodies

Fusion Protocol: Mouse Monoclonal Antibody Fusion Protocol

Monoclonal antibodies were produced from consensus sequence peptide (tetanus toxoid conjugated) by immunizing BALB/c mice using a 0, 14 and 43, day immunization schedule. The mice were boosted 4 days prior to fusion with non-conjugated consensus sequence peptide. Mouse spleen cells were fused with P3×63Ag8.653 myeloma cells (ATCC, Cat. # CRL1580) using 50% polyethylene glycol (PEG) (POLYSCIENCES, Cat. #16861) and DMEM (GIBCO, Cat #11995-073) solution, plated out in 96 well tissue culture plates (COSTAR, Cat. #3585) at 2×10⁵ cells/well and grown for one week in a 20% fetal calf serum (FCS) (HYCLONE, Cat. # SH30071.03)-DMEM (GIBCO, Cat. #11995-073) media containing 1 mM sodium hypoxanthine, 0.4 uM aminopterin, 0.016 mM thymidine media (HT-GIBCO, Cat. #31062-011). At day 5 the cells were given a feeder layer of 2×10⁶ cells/ml of BALB/c spleen cells in 20% FCS-DMEM media. On day 7, the cells were changed to a 20% FCS-DMEM media containing lmM sodium hypoxanthine, 0.016 mM thymidine (HT-GIBCO, Cat. #11067-030). Seven to ten days post-feeding, ELISA tests were done against the BSA conjugated peptide. The positive wells were selected and limiting dilutions in the presence of a BALB/c spleen feeder layer were done on each positive well. Further limiting dilutions were done after subsequent ELISA tests to obtain the antibody-producing hybridomas. Other fusion and cloning protocols known in the art may also be employed.

Monoclonal antibodies were slowly acclimatized to low FCS-DMEM. Acclimatized cells were harvested and inoculated into a CELLine device (BECTON DICKINSON, Cat. #353137) in the presence of 1% L-glutamine (GIBCO, Cat. #25030-081)-serum free media (JRH, Cat. #14620-1000M). Devices were incubated at 37° C. and 5% CO₂. Cell suspensions were harvested after seven to ten days. Suspensions were centrifuged (2000 rpm. 15 min) and filtered through 0.22 μm filter units (CORNING, Cat. #09-761-35). Supernatant was then purified. Various purification methods may be used.

In a preferred embodiment, purification of the antibody was performed by HPLC using a HiPac Protein G column (CHROMATOCHEM, Cat. # H-02-21-1000) using a Shimadzu LC 10AD. 20 mM phosphate buffer (FISHER, Cat. # BP39-500) was flushed through at 6.0 mL/minute. The syringe port was also flushed with 20 mM phosphate buffer. Antibody was injected on to the column. Phosphate buffer was run through the column for 30 minutes. The antibody was then eluted with 0.1M glycine buffer (FISHER, Cat. # G46-500). Purified antibody was collected and neutralized with 1M Tris base (BOEHRINGER MANNHEIM, Cat. #604-205). Eluting buffer was run through the column for 20 minutes followed by loading buffer for 30 minutes. Purified antibody was scanned with a spectrophotometer at 280 nm with 20 mM phosphate buffer as a blank. Final concentration (mg/mL) of the sample was determined by dividing the A₂₈₀ by 1.40.

Purified antibodies were then concentrated to 2 mg/ml using spin concentrators (CENTRICON, Cat. # UFC2LTK08). Antibodies were placed in concentrators and spun at 2700 g (4000 rpm) for 10 min. The concentrated antibody was collected and filtered through 0.22 μm filter units (CORNING, Cat. #09-761-35). A final O.D. reading was taken on the spectrophotometer at 280 nm using 20 mM phosphate as a blank. Final concentration (mg/mL) of the sample was determined by dividing the A₂₈₀ by 1.40. Antibodies were diluted to 2 mg/mL concentration by the addition of sterile 20 mM phosphate buffer if necessary. The purified antibodies were then used in characterization and passive immunization assays described below in greater detail.

Human Hybridoma Production

Monoclonal antibodies were produced from consensus sequence peptide (tetanus toxoid conjugated) immunized transgenic mice using a 0, 14 and 43-day immunization schedule. The mice were boosted 4 days prior to fusion with non-conjugated consensus sequences peptide. Mouse spleen cells were fused with P3×63Ag8.653 myeloma cells (ATCC, Cat. # CRL1580) using 50% PEG (ROCHE, Cat. #783641) and DMEM (GIBCO, Cat. #11995-073) solution, plated out in 96 well tissue culture plates (COSTAR, Cat. #3585) at 2×10⁵ cells/well and grown for one week in a 10% FCS (HYCLONE, Cat. # SH30071.03)-DMEM (GIBCO, Cat. #11995-073) media containing 1 mM sodium hypoxanthine, 0.4 uM aminopterin, 0.016 mM thymidine media (HAT-GIBCO, Cat. #31062-011) +/−2% Hybridoma Cloning factor (VWR, Cat. # CA43620-000), +/−1% OPI (SIGMA, Cat. # O-5003). On day 7, the cells were changed to a 10% FCS-DMEM media containing 1 mM sodium hypoxanthine, 0.016 mM thymidine (HT-Gibco 11067-030) +/−2% Hybridoma Cloning factor (VWR, Cat. # CA43620-000), +/−1% OPI (SIGMA, Cat. # O-5003). Three to seven days post media change, ELISA tests were performed against the BSA conjugated peptide. The positive wells were selected and limiting dilutions were conducted on each positive well. Further limiting dilutions were performed after subsequent ELISA tests to obtain the antibody-producing hybridomas.

Monoclonal antibodies were slowly acclimatized to low FCS-DMEM, 0% Hybridoma Cloning factor and 0% OPI. Cells were harvested and innoculated into a CELLine device (BECTON DICKINSON, Cat. #353137) in the presence of 1% L-glutamine (GIBCO, Cat. #25030-081)-serum free media (JRH, Cat. #14620-1000M) or BD Mab Serum Free media (BECTON DICKINSON, Cat. #220509). Devices were incubated at 37° C. and 5% CO₂. Cell suspensions were harvested after seven to ten days. Suspensions were centrifuged (2000 rpm. for 15 min) and filtered through 0.22 μm filter units (CORNING 09-761-35). Supernatant was then purified.

Purification of the antibody was performed by HPLC using a HiPac Protein G column (CHROMATOCHEM, Cat. # H-02-21-1000) using a Shimadzu LC 10AD. 20 mM phosphate buffer (FISHER, Cat. # BP39-500) was flushed through at 6.0 mL/minute. The syringe port was also flushed with 20 mM phosphate buffer. Antibody was injected on to the column. Phosphate buffer was run through the column for 30 minutes. The antibody was then eluted with 0.1 M glycine buffer (FISHER, Cat. # G46-500). Purified antibody was collected and neutralized with 1M Tris base (BOEHRINGER MANNHEIM, Cat. #604-205). Eluting buffer was run through the column for 20 minutes followed by loading buffer for 30 minutes. Purified antibody was scanned with a spectrophotometer at 280 nm with 20 mM phosphate buffer as a blank. Final concentration (mg/mL) of the sample was determined by dividing the A₂₈₀ by 1.40.

Purified antibodies were then concentrated to 2 mg/ml using spin concentrators (CENTRICON, Cat. # UFC2LTK08). Antibodies were placed in concentrators and spun at 2700 g (4000 rpm) for 10 min. The concentrated antibody was collected, filtered through 0.22 μm filter units (CORNING, Cat. #09-761-35). A final O.D. reading was taken on the spectrophotometer at 280 nm using 20 mM phosphate as a blank. Final concentration (mg/mL) of the sample was determined by dividing the A₂₈₀ by 1.40. Antibodies were brought up to 2 mg/mL concentration by the addition of sterile 20 mM phosphate buffer if necessary.

### EXAMPLE 7

Screening Techniques Anti-CS 1 Monoclonal Antibody (MoAb) In Vitro Assay Reactivity

Direct ELISA Determination of Antibody Response

Direct ELISA was used to determine positive antibodies to the consensus sequence peptide. ELISA plates (COSTAR, Cat. #3369) were coated with CS1-BSA (CYTOVAX BIOTECHINOLOGIES INC.) using a carbonate-bicarbonate buffer and incubated overnight. Following incubation, the ELISA plates were washed to remove any unbound antigen, blocking reagent (2% BSA-SIGMA, Cat. # A2153) was added and the plates were incubated to prevent any non-specific binding. The plates were then washed to remove excess blocking reagent. Individual samples from fusion plate wells were added to the ELISA wells and incubated. After incubation, the plates were washed, and an anti-mouse antibody enzyme conjugate (JACKSON LAB, Cat. #115-05-0062) was added. Following incubation, the ELISA plates were washed, and an alkaline phosphatase enzyme substrate (SIGMA Cat. #104-105) in diethanolamine buffer (Fisher Cat. # D45-500) was added. Once the enzyme reaction had developed, the absorbance was determined using a plate reader (MOLECULAR DEVICES). Positive antibodies were expressed as optical density (O.D.) versus dilution.

Isotyping Antibody Response to the Consensus Sequence by ELISA Assay

An isotyping ELISA was used to determine isotype of polyclonal and monoclonal antibodies to the consensus sequence peptide. Antibody response to TI and TD antigens differ. In the mouse, the response to a polysaccharide or small peptide antigen (TI antigen) is usually composed of a one-to-one ratio of IgM and IgG. In general, IgG isotypes are restricted, wherein IgG₃ is over-expressed in anti-polysaccharide serum. IgA isotypes may also be present. TI antigens elicit antibodies with low affinity and immunologic memory is not produced.

With TD antigens, increased secondary IgG antibody responses (an anamnestic response) are found, with a higher IgG to IgM ratio. Marked levels of IgA are usually not present. The TD antigen elicits a heterogeneous IgG isotype response, wherein the predominant isotype is IgG₁, IgG₂ₐ and IgG_{2b} isotypes can be expressed, while the IgG₃ isotype level is usually relatively low. TD antigens elicit immunologic memory, and antibody affinity increases with immunizations. Thus, analysis of the immunoglobulin isotypes produced in response to conjugate administration enables one to determine whether or not a conjugate will be protectively immunogenic.

Applicants have found that the conjugates of the present invention can induce a response typical of TD, rather than TI antigens, as measured by direct and isotyping ELISA and opsonization assay.

ELISA plates (COSTAR, Cat. #3369) were coated with the CS1-BSA antigen (CYTOVAX BIOTECHNOLOGIES INC.) using a carbonate-bicarbonate buffer and incubated overnight. Following incubation, the ELISA plates were washed to remove any unbound antigen, blocking reagent (2% BSA-SIGMA, Cat. # A2153) was added, and the plates were incubated to prevent any non-specific binding. The plates were then washed to remove excess blocking agent. Cell culture supernatant samples were added to the ELISA wells and incubated. After incubation, the plates were again washed and either anti-mouse IgM, IgG₁, IgG₂ₐ, IgG_{2b} or IgG₃ antibody enzyme conjugates (SBA Clonotyping™ Systems, Cat. #5300-05) or anti-human antibody enzyme conjugates IgG₁ (ZYMED Cat. #05-3300), IgG₂ (ZYMED Cat. #05-3500), IgG₃ (ZYMED Cat. #05-3600), IgG₄ (ZYMED Cat. #05-3800) or IgM (ZYMED Cat, #05-4900) were added. After incubation, the plates were washed and an ABTS (2,2′-azinobis(3-ethylbenzothiazoline)-6 sulfonic acid diammonium salt (ROCHE DIAGNOSTICS, Cat. #102 946) enzyme substrate was added. The plates were allowed to develop and the absorbance was measured using a plate reader (MOLECULAR DEVICES). Typical results of production of mouse MoAb are shown below in Table 6. Human MoAb production results are shown below in Table 7.

TABLE 6
Mouse MoAb Isotyping to Consensus Sequence (CS1)

| Isotype | O.D. |
|---|---|
| IgG1 | 4 |
| IgG2a | 0.1945 |
| IgG2b | 0.1559 |
| IgG3 | 0.159 |
| IgM | 0.119 |

TABLE 7
Human MoAb Isotyping to CS1

| Isotype | O.D. |
|---|---|
| IgG1 | 3.53 |
| IgG2 | 0.42 |
| IgG3 | 0.37 |
| IgG4 | 0.21 |
| IgM | 0.63 |

### EXAMPLE 8

Antigen Panel ELISA Assay to Measure Cross-Reactivity

Direct antigen panel ELISA assay was used to determine the cross-reactivity of the anti-consensus sequence peptide polyclonal and monoclonal antibody to pilin peptide cell-binding domains of Pseudomonas aeruginosa (PAO: SEQ ID NO: 2; PAK: SEQ ID NO: 1; KB7: SEQ ID NO: 11; and K122-4: SEQ ID NO: 10). ELISA plates (COSTAR, Cat. #3369) were coated with antigens (BSA, PAO-BSA, PAK-BSA, CS1-BSA, K122-4-BSA, P1-BSA and KB7-BSA, CYTOVAX BIOTECHNOLOGIES INC.) using a carbonate-bicarbonate buffer and incubated overnight. Following incubation, the ELISA plates were washed to remove any unbound antigen, 2% BSA blocking reagent (SIGMA, Cat. # A2153) was added and the plates were incubated to prevent any non-specific binding. The plates were then washed to remove excess blocking agent. Diluted test samples were added to the ELISA wells and incubated. After incubation, the plates were washed and anti-mouse antibody enzyme conjugate (JACKSON LAB, Cat. #115-035-062) was added. After incubation, the ELISA plates were washed and an ABTS enzyme substrate was added. Once the enzyme reaction had developed, the absorbance was read using a plate reader (MOLECULAR DEVICES).

The anti-CS 1 polyclonal antibody showed cross-reactivity to native pilin sequence peptides derived from different strains of Pseudomonas aeruginosa (PAK, PAO, KB7, and K122-4) as illustrated below in Table 8. There was no non-specific binding to the BSA coated ELISA wells.

TABLE 8
Polyclonal Cross-Reactivity to Clinical Strains of
Pseudomonas Aeruginosa

| CS1-TT | O.D. |
|---|---|
| CS1-BSA | 3.840 |
| PAK-BSA | 3.773 |
| PAO-BSA | 3.553 |
| KB7-BSA | 3.055 |
| K122-4 | 2.570 |
| BSA | 0.157 |

### EXAMPLE 9

Western Blot Analysis of Anti-CS1 MoAb to Various Bacterial Cell Extracts and Recombinant Pilin Protein

Anti-CS1 monoclonal antibody was diluted in PBS. Equal parts of antibody and sample buffer (2-mercaptoethanol (BIO-RAD, Cat. #161-0710) diluted 1:20 with Laemmli Sample Buffer (BIO-RAD, Cat. #161-0737)) were combined and boiled for 10 minutes. 12% resolving gels (BIO-RAD, Cat. #161-1156) were assembled onto gel apparatus (BIO-RAD). After washing wells with running buffer (1.9 M Glycine (FISHER, Cat. # G46-500), 0.25 M Tris Base (FISHER, Cat. # BP152-1) and 35 μM Sodium dodecyl sulphate (FISHER, Cat. # BP166-500)), antibody and standard (INVITROGEN, Cat. # LC5625) were loaded onto wells. The gel was run at 100V. Gels were then transferred into a transfer cassette containing a nitrocellulose membrane (BIORAD, Cat. #162-0115) and run at 100 V for 1 hour. The membrane was blocked for 1 hour at 37° C. and washed three times with TTBS (0.1% Polyoxyethylene Sorbitan Monolaurate, SIGMA, Cat. # P7949), 0.05 M Tris Base (FISHER, Cat. # BP152-1) and 0.14M NaCl (FISHER, Cat. # S271). Secondary antibody (JACKSON LABORATORIES, Cat. #115-055-062) was added to the membrane and incubated 1 hour at 37° C. The membrane was washed twice with TTBS and once with TBS (0.05 M Tris Base (FISHER, Cat. # BP152-1)), 0.14M NaCl (FISHER, Cat. # S271). Substrate NBT/BCIP (ROCHE DIAGNOSTICS, Cat. #169471) was added to membrane and allowed to develop. The membrane was rinsed with distilled water to stop the reaction. It was then air-dried.

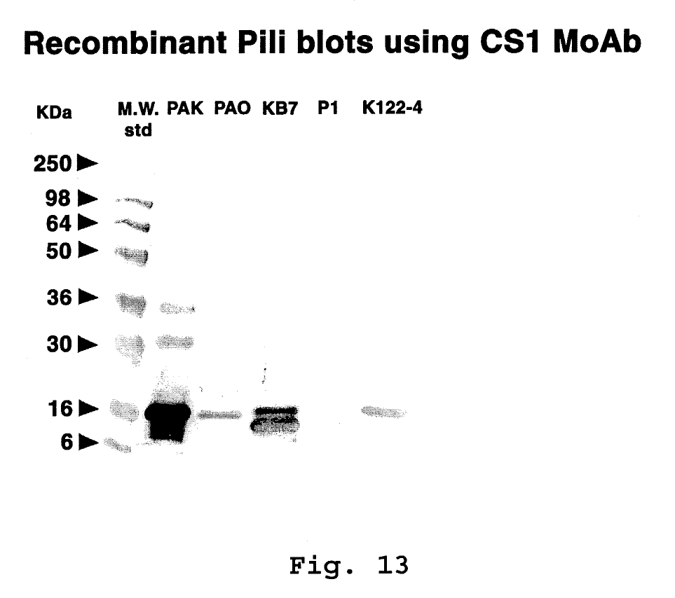

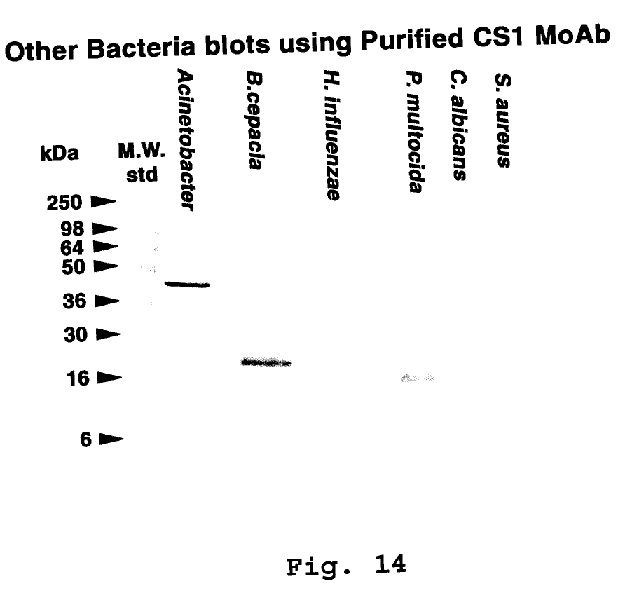

The results of the Western Blot Analysis are shown in FIGS. 13 and 14. The anti-CS1 MoAb recognized a 14-16 Kda band of Pseudomonas aeruginosa whole cell extracts (PAK: SEQ ID NO: 1, PAO: SEQ ID NO: 2, P1: SEQ ID NO: 12, KB7: SEQ ID NO: 11, K122-4: SEQ ID NO: 10). This MoAb also specifically bound to recombinant pilin protein from PAK: SEQ ID NO: 1, PAO: SEQ ID NO: 2; KB7: SEQ ID NO: 11, P1: SEQ ID NO: 12 and K122-4: SEQ ID NO: 10 as shown in FIG. 13. The anti-CS1 MoAb cross-reacted with various other bacterial strains including but not limited to Acinetobacter spp., Burkholderia cepacia, Haemophilus influenza and Pasteurella multiocida as shown in FIG. 14.

### EXAMPLE 10

BIACORE

The cross-reactivity of anti-CS1 with various pilin peptides was measured in real time using a BIACORE 3000 biosensor system. The system reports changes in refractive index at the surface of the biosensor chip by detecting changes in the angle of incident light at which surface plasmon resonance occurs. The principle and application of SPR detection to monitor biomolecular interactions have been described previously (See, Jonsson et al., (1991) Biotechniques 11, 620-627; and Karlsson et al., (1991) J. Immunol. Methods 145, 229-240).

Individual pilin peptides were coupled to BSA in a two-step process. The peptides were first iodoacetylated at their N-terminus with iodoacetic anhydride at pH 6.0. BSA was treated with Traut's reagent that alkylates lysine residues and subsequently generates a free thiol. The thiols on BSA serve as an attachment site for the peptides by reacting with the iodoacetylated N-terminus, thus generating a thioether bond. The pilin peptide/BSA conjugates or BSA (as a negative control chip) are subsequently attached to a carboxymethylated BIACORE chip using standard NHS/carbodimide chemistry as outlined in the BIACORE manual.

For initial kinetic measurements, each BSA conjugate (20 μg/mL) was injected onto the activated surface of the chip over 1 minute at a flow rate of 5 μl/min. This yielded a thereotical Rmax of approximately 500 to 1000 RU. A stock solution of anti-CS1 MoAb (0.25 μM) was flowed over the chip at a flow rate of 35 μL/min and the interaction was measure by SPR. Anti-CS1 MoAb was then serially diluted and flowed over the chip surface. The BIACORE 3000 software calculated various kinetic parameters outlined in Table 9 as shown below. These experiments were run again with the various pilin/BSA conjugates and the results tabulated below.

TABLE 9
BIACORE Analysis

| BSA-Conjugate | kₐ (1/Ms) | k_{d} (l/s) | K_{D} (M) |
|---|---|---|---|
| CS1 | 1.1 × 10⁴ | <1 × 10⁻⁵ | 9.4 × 10⁻¹⁰ |
| PAK | 3.8 × 10⁴ | <1 × 10⁻⁵ | 2.7 × 10⁻¹⁰ |
| PAG | 4 × 10⁴ | 7.5 × 10⁻⁴ | 1.9 × 10⁻⁸ |
| P1 | 1.8 × 10³ | 5.6 × 10⁻³ | 3.6 × 10⁻⁶ |
| K122-4 | 1.6 × 10⁵ | >0.1 | 6.1 × 10⁻⁷ |

Based on these results, the clone binds PAK slightly better than CS1 and both have equilibrium dissociation constants in the high pM range. The interaction of the MoAb with P1 is not as strong as with CS1 and PAK. The rate constant for the interaction of Anti-CS1 MoAb with P1 chip is about twenty-one times lower than the interaction of the MoAb with PAK. The dissociation rate constant is about 560 times higher compared with dissociation of MoAb from the PAK surface. The equilibrium dissociation constant for P1 is in the low μM range.

With K122-4, the MoAb interaction is slightly different. The MoAb interacts with the K122-4 chip at rates similar to CS1 and PAK, but dissociates very quickly. However, these results show that there is significant binding to all the major pilin peptide groups of Pseudomonas aeruginosa.

This BIACORE data demonstrates kinetic antibody binding evidence that the anti-CS1 MoAb binds PAK, PAO, KB7, P1, K122-4 pilin peptides (90% of the clinically relevant Pseudomonas aeruginosa strains). Various other antigens including but not limited to recombinant pilin protein could be bound to the BIACORE chip for analysis.

### EXAMPLE 11

Opsonization Assay as a Measure of Antibody Phagocytic Protection Against Various Pseudomonas Bacteria Strains

To a sterile flat bottom 96 well plate with a sterile 2.5 mm glass bead (VWR 26396-634) in each well (triplicates): 5 μl of Pseudomonas aeruginosa, 50 μl of Heparinized Blood and 10 μ of monoclonal antibody were added as well as media and complement controls. The plates were incubated at 37° C. for 1 hour on a shaker (slow motion). A 50 μl aliquot was plated out on a TSB (Tryptic Soy Broth—DALYNN LABS, Cat. # PT80-500) Petri plates. The plates were wrapped and incubated at 37° C. for 12 hours. The colony forming units on each plate were counted and reported as Percent Inhibition of CFU's.

TABLE 10
Opsonization Assay: Mouse CS1 MoAb

| Pseudomonas Strains | Percent Inhibition |
|---|---|
| PAK | 83.6 |
| PAO | 86.2 |
| KB7 | 40 |
| P1 | 75.6 |
| K122-4 | 54.3 |
| Positive control | 100 |
| Media | 0 |

The mouse CS1 MoAb exhibited opsonic activity to various strains of P. aeruginosa. This assay is generally considered a reliable indication of immunoprotective capability in vivo.

### EXAMPLE 12

Adhesion Inhibition Assays

The purpose of this study was to evaluate antibody inhibition of Pseudomonas aeruginosa pilin protein adhesion to a human epithelial cell line (A549).

Anti-CS1 monoclonal antibody inhibited adhesion of recombinant pilin protein to the human epithelial cell line. The negative control monoclonal antibody did not inhibit adhesion. Anti-CS1 mouse sera also inhibited binding of Pseudomonas aeruginosa pilin protein to the epithelial cell line wherein the anti-TT mouse sera did not inhibit.

A stock solution of 10 mg of biotinamidocaproate N-hydroxysuccinimide ester (SIGMA, Cat. # B2643) was dissolved in 0.5 mL of DMSO (Dimethyl Sulfoxide—SIGMA, Cat. # D8779). Thirty μL of this stock solution was added to 1.0 mL of 1 mg/mL recombinant pilin protein suspended in PBS and incubated for 2 hours at room temperature. The solution was constantly shaken using a gyro-shaker (LAB-LINE INSTRUMENTS) and 10 mM Glycine (SIGMA Cat. # G46-500) was added to quench the reaction. The quenched solution was placed into a dialysis cassette and dialyzed extensively at 4° C. against 4 changes of PBS (2 L each). The solution was removed from the cassette and the ability of the biotinylated-pili to bind to human alveolar epithelial A549 cells (ATCC Cat. # CLL185) was tested and confirmed. A549 cells were grown at 37° C., 5% CO₂ and maintained in 25 cm² tissue culture flasks (CORNING, Cat. #10-126-28) containing Ham's F12K medium (GIBCO, Cat. #11765-054) supplemented with 10% (v/v) heat-inactivated fetal calf serum (HYCLONE, Cat. # SH30071.0) and 100 units/mL of penicillin and streptomycin (GIBCO, Cat. #15140-022). To harvest cells, the cell monolayer was detached by a 5 minute incubation at 37° C. in a trypsin-EDTA/PBS solution (0.05% w/v trypsin—SIGMA Cat. # T4799-0.53 mM EDTA—SIGMA Cat. # E6635). The harvested cells were suspended in Ham's F12K culture medium followed by centrifugation at 1500 rpm for 5 min at room temperature. Supernatant was removed, cells were resuspended and the centrifugation step was repeated. The cells were resuspended in medium and adjusted to 4×10⁵ cells/mL. A 100 μL aliquot of cell suspension was seeded in 96-well tissue culture plates (COSTAR Cat. #3585) and incubated at 37° C. overnight. On the following day, the medium was removed from each well by aspiration and wells were gently washed with 200 μL of medium.

Biotinylated pili stock was diluted to a concentration of 1 ng/mL in culture medium containing 0.02% sodium azide (SIGMA Cat. # S227-25) and mixed with serially diluted monoclonal antibody or anti-CS1 mouse sera. A positive binding control, medium containing biotinylated pili and no antibody, to confirm binding capacity of the biotinylated pili was included. Antibody plus pilin mixtures (75 μL) were added to the plate wells and incubated at 37° C. for 2 hours. The plates were washed three times with 250 μL per well of culture medium and once with 250 μL per well of HBSS (Hanks Balanced Salts—SIGMA Cat. # H1387). Cell monolayers were fixed by addition of 100 μL of 0.25% glutaraldehyde (SIGMA Cat. # G6257) in HBSS per well and incubated at 37° C. for 1 hour. The plates were washed three times with 250 μL per well of HBSS, and the unreacted glutaraldehyde was neutralized by incubation at 37° C. for 1 hour with 250 μL per well of 50 mM glycine (SIGMA Cat. # G46-500). The neutralizing solution was aspirated, and the plates washed twice with 250 μL per well of HBSS. Streptavidin-horseradish peroxidase (75 μL, SIGMA Cat. # S9420) in PBS/1% BSA (SIGMA Cat. # A2153) was added to each well. Plates were incubated at 37° C. for 1 hour and washed four times with 200 μL per well of PBS/0.05% BSA (SIGMA Cat. # A2153). The substrate, OPD (o-Phenylethylenediamine dihydrochloride—SIGMA Cat. # P1526) in citrate phosphate buffer was added and plates were allowed to develop.

The reaction was quenched with 1N HCl and the absorbance was measured at 490 nm using a VERSAMAX plate reader (MOLECULAR DEVICES). Absorbance readings of the positive binding controls provided a measure of 0% inhibition of binding of pili. Readings obtained with test antibodies provided a measure of the degree of inhibition of pili binding.

The following table demonstrates typical adhesion assay results obtained with anti-CS1 MoAb or mouse sera.

TABLE 11
In Vitro Pilin Adhesion Assay using Mouse sera to the Consensus
Sequence

| Mouse Sera | Percent Inhibition |
|---|---|
| Anti-CS1 TT 1:10 | 66.4 |
| Anti-CS1 TT 1:20 | 51.7 |
| Anti-CS1 TT 1:40 | 30.1 |
| Anti-CS1 TT 1:80 | 1.1 |
| Anti-TT 1:20 | 13.3 |
| Anti-TT 1:40 | 3.7 |
| Anti-TT 1:80 | 0 |
| Anti-TT 1:10 | 0 |

TABLE 12
In Vitro Pilin Adhesion Assay using Anti-CS1 MoAb

| Mouse Sera | Percent Inhibition |
|---|---|
| Anti-CS1 Mab 10 μg | 91.0 |
| Anti-CS1 Mab 3 μg | 75.6 |
| Anti-CS1 Mab 1 μg | 60.6 |
| Anti-CS1 Mab .3 μg | 36.5 |
| Negative Control MoAb 10 μg | 0.1 |

Pilin protein binds to receptors on human epithelial cell line. The pilin adhesion assay tests the ability of anti-CS1 antibodies to inhibit pilin binding to an epithelial cell line. Anti-CS1 monoclonal antibody or anti-CS1 mouse sera inhibited pilin binding to human epithelial cell line. This inhibition was antibody dose dependent. The negative control monoclonal antibody and anti-TT mouse sera did not block pilin binding. These in vitro assay results demonstrate the principle of the anti-adhesion property of anti-CS1 antibodies.

### EXAMPLE 13

IEF

Isoelectric focusing was used to check purity of the Anti-CS1 monoclonal antibody. 5 μg of purified antibody and 1 μl of IEF standard (BIORAD, Cat. #161-0310) were run on an IEF agarose gel (BIOWHITTAKER MOLECULAR APPLICATIONS, Cat. #56016). The flatbed assembly was wet, and the agarose gel was placed on it. Two wicks were soaked in either anode solution (0.5 M acetic acid) or cathode solution (0.5 M NaOH). An application mask (BIOWHITTAKER MOLECULAR APPLICATIONS, Cat. #56014) was placed at the gel center and 5 μL of antibody, and 1 μL standard were applied to well and left to diffuse for 5 minutes. The wicks were blotted and placed horizontally parallel to the gel edges on the anodal and cathodal ends respectively. Electrode assembly was adjusted to ensure contact between wires and wicks. Tap water was connected to the flatbed system to maintain temperature at approximately 15° C. The system was connected to a Power Pac 3000 (BIORAD) and set to 1W and 20 mA at 1200V and run 10 minutes to allow sample absorption. The system was then set to 25W and run for 50 minutes (same voltage and mA). The gel was fixed for ten minutes in fixing buffer −0.367M Trichloroacetic acid (FISHER, Cat. # A322-100), 0.142M sulfosalicyclic acid (SIGMA, Cat. # S3147) in 38% Methanol: double distilled H₂O (FISHER, Cat. # A452-4). After fixing, the gel was rinsed with distilled water and placed in 95% Methanol (FISHER, Cat. # A452-4) for 30 minutes. The gel was blotted dry, rinsed with distilled water and hung to dry completely. The dried gel was gently agitated for 15 minutes in staining solution (BIORAD, Cat. #161-0434). It was then rinsed with distilled water and placed in destain solution (BIORAD, Cat. #161-0438) until background appeared clear. The gel was rinsed with distilled water and hung to dry. This method was used to monitor the protein purity and to detect any alterations of the protein form. A consistent and clean band pattern was shown between isoelectric points 5.1-6.0.

### EXAMPLE 14

A Murine Model for Pseudomonas Infection

The purpose of this study was to evaluate the immunogenic efficacy of CS1-TT conjugate to protect against a lethal challenge of Pseudomonas aeruginosa. It was demonstrated that immunizations with CS1-TT conjugate elicited protective antibodies against various Pseudomonas aeruginosa strains.

Three strains of Pseudomonas aeruginosa (PAK, PAO, P1) were used in these challenge studies. These strains were originally isolated from clinical patients. The organisms were stored at −80° C. and passaged on fresh tryptic soy agar (DIFCO, Cat. # BP1423-2) plates weekly. For challenge, a single colony of Pseudomonas aeruginosa was used to inoculate 10 mL of tryptic soy broth (DIFCO, Cat. #211822) and grown overnight at 37° C. The next day, an aliquot(s) of the overnight suspension was transferred to 10 mL of fresh tryptic soy broth in a side-arm flask until an optical density of 0.04 (560 nm) was achieved (BIOCHROM ULTRASPEC 2100 pro spectrophotometer, FISHER SCIENTIFIC, Cat. # BC80-2112-21). This culture was then incubated at 37° C. and shaken at 160 rpm with a Lab Companion SI-600 incubator/shaker (ROSE SCIENTIFIC, Cat. #3527). When the appropriate O.D. value of the culture was obtained, the concentration was diluted to achieve the required challenge dose. Serial dilutions of the challenge culture were streaked onto tryptic soy agar plates to confirm the number of Colony Forming Units/mL (CFU/mL).

Groups of A.BY mice (10 per group) were immunized with CS1-TT or scrambled peptide-TT (negative control). On day 62, mice (final weight between 20-27 g) were i.p. challenged with a 100 μL lethal dose (˜LD₈₀) of Pseudomonas aeruginosa (PAO challenge dose, 2.5×10⁵ CFU/mouse; PAK challenge dose, 1.0×10⁶ CFU/mouse; P1 challenge dose, 1.0×10⁶ CFU/mouse). Mice were monitored for relative degrees of morbidity and mortality associated with systematic infection. This was accomplished by observing challenged mice once per hour for 48 hours (PAK and P1) or for 65 hours (PAO). Symptom monitoring began at 14 hours after challenge for signs of infection or illness. The potential symptoms include; a hunched back, ruffled fur, lethargy, cyanosis, dehydration, conjunctivitis, mucous diarrhea, emaciation or loss of righting reflex. Once the righting reflex was lost, the animal was euthanized with the time of death recorded. The survival time for each mouse was recorded.

The following are examples of survival curves in this A.BY challenge model (Table 13, see below). Mean survival times and P values are also shown (statistical program—Systat 9). These results show that mice immunized with CS1-TT absorbed onto alum demonstrated good protection (about 60 to about 100% survival) to a lethal challenge dose of Pseudomonas aeruginosa (PAK, PAO, and P1). The survival rate of mice immunized with the scrambled peptide-TT absorbed onto alum (negative control) was only about 0 to about 10%. These challenge model results were reproducible.

TABLE 13
Pseudomonas aeruginosa in A.BY Mouse Challenge Model

| Groups | PAO | PAK | P1 |
|---|---|---|---|
| CS1-TT | | | |
| Percent survival | 100 | 60 | 60 |
| Mean Survival time | 65 | 40.5 | 47.2 |
| Scrambled Peptide | | | |
| -TT | | | |
| Percent survival | 0 | 0 | 10 |
| Mean Survival time | 26.9 | 20.7 | 23.7 |
| P value | <0.0001* | <0.0045* | <0.0051* |

*indicates that the survival curves are significantly different

These results demonstrate that a consensus sequence immunogen can elicit protection to multiple strains of Pseudomonas aeruginosa.

### EXAMPLE 15

Ex Vivo Monoclonal Antibody Neutralization

The purpose of this study was to evaluate the ability of an anti-CS1 monoclonal antibody to neutralize various strains of Pseudomonas aeruginosa in an ex vivo challenge model. Anti-CS1 monoclonal antibody neutralized Pseudomonas aeruginosa strains PAO, PAK, KB7, K122-4 and P1. The negative control monoclonal antibody showed no neutralization ability.

A breeding colony of BALB/c mice at the Health Sciences Laboratory Animal Services (University of Alberta) provided the mice used in this study. Female BALB/c mice were received at 6-8 weeks of age. The mice were housed in polycarbonate rodent cages (NALGENE, Cat. #01-288-1C) and fed certified autoclavable mouse diet (PURINA, Cat. #5010) and deionized autoclaved water. All animal studies complied with the guidelines established by the Canadian Council on Animal Care and the requirements of the Health Sciences Animal Policy and Welfare Committee at the University of Alberta. Environmental parameters of the animal room were monitored using a data logger. The light cycle was maintained at 12 hours light and 12 hours dark. Temperature was maintained at 22° C. (±2° C.) and humidity was maintained between 40% and 70% relative humidity.

Five strains of Pseudomonas aeruginosa (PAK, PAO, P1, KB7 and K122-4) were used in these challenge studies. These strains were originally isolated from clinical patients. The organisms were stored at −80° C. and passaged on fresh tryptic soy agar (DIFCO, Cat. # BP1423-2) plates weekly. For challenge, a single colony of Pseudomonas aeruginosa was used to inoculate 10 mL of tryptic soy broth (DIFCO, Cat. #211822) and grown overnight at 37° C.

The next day an aliquot(s) of the overnight suspension was transferred to 10 mL of fresh tryptic soy broth in a side-arm flask until an optical density of 0.04 (560 nm) was achieved (BIOCHROM ULTRASPEC 2100 pro spectrophotometer, FISHER SCIENTIFIC, Cat. # BC80-2112-21). This culture was then incubated at 37° C. and shaken at 160 rpm with a LAB COMPANION SI-600 incubator/shaker (ROSE SCIENTIFIC, Cat. #3527). When the appropriate O.D. value of the culture was obtained the concentration was diluted to achieve the required challenge dose. Serial dilutions of the challenge culture were streaked onto tryptic soy agar plates to confirm the number of CFU/mL.

The anti-CS1 monoclonal antibody, (IgG1 isotype), or a negative control antibody, MOPC 31c (IgG₁ isotype, ATCC Cat. # CLL-130), were combined with various Pseudomonas aeruginosa strains and then incubated for one hour at 37° C. The pre-incubation consisted of a 600 μL aliquot of various strains of Pseudomonas aeruginosa, and a 600 μL aliquot (2 mg/mL) of either anti-CS1 MoAb or MOPC 31c. The Pseudomonas aeruginosa strains used in this study were used at the appropriate challenge dose (PAO challenge dose, 5.5×10⁵ CFU/mouse; PAK challenge dose, 2.0×10⁶ CFU/mouse; KB7 challenge dose, 2.0×10⁶ CFU/mouse; P1 challenge dose, 2.0×10⁶ CFU/mouse and K122-4 challenge dose, 4.0×10⁶). Following the incubation, BALB/c mice (final weight between 20-27 g) were challenged i.p. with a 100 μL aliquot of the bacteria/monoclonal antibody mixture.

Mice were monitored for relative degrees of morbidity and mortality associated with systematic infection. This was accomplished by observing challenged mice once per hour for 48 hours (PAK, KB7, K122-4 and P1) or for 65 hours (PAO). Symptom monitoring began at 14 hours after challenge for signs of infection or illness. The potential symptoms include; a hunched back, ruffled fur, lethargy, cyanosis, dehydration, conjunctivitis, mucous diarrhea, emaciation or loss of righting reflex. Once the righting reflex was lost, the animal was euthanized with the time of death recorded. The survival time for each mouse was recorded.

As illustrated below, Table 14 shows survival results obtained with this ex vivo BALB/c challenge model. Mean survival times and P values are also shown. The results demonstrate that the anti-CS1 MoAb neutralizes various strains of Pseudomonas aeruginosa (PAK, PAO, KB7, K122-4 and P1). This study showed an about 80 to 100% survival rate in the mouse group receiving the anti-CS1 MoAb while the mouse group receiving the negative control MoAb had a low survival rate of about 0 to 30%.

TABLE 14
Pseudomonas aeruginosa Strain in the Ex Vivo Neutralization Study

| Groups | PAK | PAO | KB7 | P1 | K122-4 |
|---|---|---|---|---|---|
| CS1 MoAb | | | | | |
| Percent | 100 | 100 | 100 | 80 | 90 |
| survival | | | | | |
| Mean | 48.0 | 48.0 | 48.0 | 41.9 | 47.2 |
| Survival | | | | | |
| time | | | | | |
| MOPC | | | | | |
| MoAb | | | | | |
| Percent | 0 | 30 | 30 | 30 | 0 |
| survival | | | | | |
| Mean | 15.0 | 27.0 | 25.6 | 25.6 | 17.7 |
| Survival | | | | | |
| time | | | | | |
| P value | <0.0001* | <0.0002* | <0.0014* | <0.0059* | <0.0001* |

*indicates that the survival curves are significantly different

The survival curves from anti-CS1 treated mice compared with the survival curves of MOPC 31c treated mice were statistically significant as determined by P values (PAO, P value=0.0002; PAK, P value<0.0001; KB7, P value=0.0014, K122-4; P value<0.0001, P1, P value=0.0059). These ex vivo assay results were reproducible. These results demonstrate that an anti-consensus sequence MoAb can neutralize multiple strains of Pseudomonas aeruginosa. The consensus sequence immunogen can elicit neutralizing antibody to about 90% of the clinically relevant Pseudomonas aeruginosa bacterial strains.

### EXAMPLE 16

Monoclonal Antibody Passive Model

The purpose of this study was to evaluate the protection provided by passively administering anti-CS 1 MoAb in a lethal dose BALB/c challenge model.

It was concluded that passive immunization with anti-CS1 MoAb showed protection against Pseudomonas aeruginosa strains PAO, PAK, KB7 and P1. The negative control monoclonal antibody (MOPC 31c) showed no protective effect

The monoclonal antibody used in this study (anti-CS1 MoAb) was developed from CS1 peptide sequence as outlined in Example 13 discussed above.

BALB/c mice (final weight between 20-27 g) were i.v. administered anti-CS1 MoAb or MOPC 31c (10 mg/kg dose) at 24 and 8 hours prior to bacterial challenge. The mice were then i.p. challenged with lethal doses of Pseudomonas aeruginosa strains (PAO, PAK, KB7 and P1, lethal CFU dosage). Mice were monitored for relative degrees of morbidity and mortality associated with systematic infection. This was accomplished by observing challenged mice once per hour for 48 hours (PAK, KB7 and P1) or for 65 hours (PAO). Symptom monitoring began at 14 hours after challenge for signs of infection or illness. The potential symptoms include: a hunched back, ruffled fur, lethargy, cyanosis, dehydration, conjunctivitis, mucous diarrhea, emaciation or loss of righting reflex. Once the righting reflex was lost, the animal was euthanized with the time of death recorded. The survival time for each mouse was recorded.

Data in the Table 15 below demonstrates that anti-CS1 MoAb protected against a lethal dose challenge with various strains of Pseudomonas aeruginosa (PAK, PAO, KB7, and P1). The anti-CS1 monoclonal group showed about 60 to about 100% survival rate while the negative control group had a low survival rate of about 0 to about 20%.

TABLE 15
Anti-CS MoAb in the Passive Model

| Groups | PAK | PAO | KB7 | P1 | K122-4 |
|---|---|---|---|---|---|
| CS1 MoAb | | | | | |
| Percent | 90 | 60 | 100 | 90 | 60 |
| survival | | | | | |
| Mean | 45.2 | 37.2 | 48.0 | 44.9 | 41.8 |
| Survival | | | | | |
| time | | | | | |
| MOPC | | | | | |
| MoAb | | | | | |
| Percent | 0 | 0 | 20 | 20 | 10 |
| survival | | | | | |
| Mean | 14.5 | 16.8 | 14.3 | 22.8 | 32.7 |
| Survival | | | | | |
| time | | | | | |
| P value | <0.0001* | <0.0037* | <0.0001* | <0.0018* | <0.0246 |

*indicates that the survival curves are significantly different

The survival curves of mice passively administered anti-CS1 MoAb compared to survival curves of mice passively administered MOPC 31c were statistically significant as determined by P values (PAO, P value=0.00037; PAK, P value=<0.0001; KB7, P value=<0.0001; P1, P value=0.0018). These passive immunization results were reproducible.

### EXAMPLE 17

Monoclonal Antibody Post Challenge Passive Model

The purpose of this study was to evaluate the protection provided by administering anti-CS1 MoAb post-challenge in a lethal dose BALB/c challenge model. It was concluded that post-challenge immunization with anti-CS1 MoAb 39 showed protection against Pseudomonas aeruginosa strain PAK. The negative control monoclonal antibody (MOPC 31c) showed no protective effect. The monoclonal antibody used in this study, anti-CS1 MoAb was developed from CS1 peptide sequence as outlined in Example 6.

BALB/c mice (final weight between 20-27 g) were i.p. challenged with lethal doses of Pseudomonas aeruginosa strains (PAO, PAK, KB7 and P1, CFU's as previously described). The mice were then i.v. administered anti-CS1 MoAb or MOPC 31c (10 mg/kg dose) at 1,2 and 4 hours post bacterial challenge. Mice were monitored for relative degrees of morbidity and mortality associated with systematic infection. This was accomplished by observing challenged mice once per hour for 48 hours (PAK, KB7 and P1) or for 65 hours (PAO). Symptom monitoring began at 14 hours after challenge for signs of infection or illness. The potential symptoms include: a hunched back, ruffled fur, lethargy, cyanosis, dehydration, conjunctivitis, mucous diarrhea, emaciation or loss of righting reflex. Once the righting reflex was lost, the animal was euthanized with the time of death recorded. The survival time for each mouse was recorded.

The following Table 16 demonstrates that anti-CS1 MoAb protected against a lethal dose challenge with Pseudomonas aeruginosa strain (PAK). The anti-CS1 monoclonal group showed about 60 to about 100% survival rate while the negative control group had a low survival rate of about 0 to about 20%.

TABLE 16
A.BY Passive Immunization Results

| | Post 1 hour | Post 2 hours | Post 4 hours |
|---|---|---|---|
| | 1.27 × 10⁶ | 0.55 × 10⁶ | 0.31 × 10⁶ |
| Groups | cfu/Mouse | cfu/Mouse | cfu/Mouse |
| CS1 MoAb | | | |
| Percent survival | 60 | 70 | 80 |
| Mean Survival time | 40.0 | 40.0 | 40.2 |
| MOPC MoAb | | | |
| Percent survival | 20 | 20 | 20 |
| Mean Survival time | 29.1 | 31.0 | 27.0 |
| P value | <0.0222* | <0.0350* | <0.0076* |

*indicates that the survival curves are significantly different

### EXAMPLE 18

Active Intratracheal Model

CF mice received from Case Western University were acclimatized for one week before surgery. Mice were immunized intranasally (i.n.) on day 0, 7 28, and 42. On day 60, mice were challenged intratracheally (i.t.). Mice were weighed the day of surgery and pre-medicated with Buprenorphine (RECKITT & COLEMAN, Inc.) 0.1 mg/kg s.c. for intra-operative and post-operative analgesia. Mice were anesthetized with a cocktail of Ketamine (BIMEDA-MTC INC.) 75 mg/kg, and Acepromazine (AYERST, Inc.) 2.5 mg/kg injected i.p. Anesthetic cocktail: 0.72 mL Ketamine (BiMeda-MTC, Inc.), 0.24 mL Acepromazime (Ayerst, Inc), and 8.64 mL sterile PBS were mixed into a sterile 10 mL capped vial. Anesthetic cocktail was made fresh the morning of surgery. NAIR depilatory cream (CARTER-HOMER, INC.) was applied to the throat area for 10 minutes to remove hair from the surgical site.

After 10 minutes, the NAIR and hair are wiped away with 2×2 gauze (JOHNSON & JOHNSON). Once the mouse was in surgical plane anesthesia, it was placed in dorsal recumbancy. A piece of 3/0 silk suture (ETHICON, INC.) was used to lasso the upper incisors and secure the head in a steady position. The tail was secured with masking tape. The surgical site was prepped with proviodine. A 1 cm skin incision was made on the ventral cervical surface cranial to the thoracic inlet of the trachea. The subcutaneous fat and muscle was reflected using smooth forceps and blunt dissection. Muscle and fascia was dissected from the trachea such that it could be clearly seen. A 24 g×¾ inch venocatheter (JORVET, INC.) was used to cannulate the trachea. Caudal to the larynx, the needle was inserted bevel up into the lumen of the trachea.

After introduction of the catheter, the needle was removed, and the catheter was advanced to a point above the bifurcation of the trachea. A 1 mL syringe was filled to 200 μL with the inoculum (Pseudomonas aeruginosa agarose beads). The syringe was attached to the hub of the catheter. Since the hub and lumen of the catheter hold 50 μL, 100 μL of inoculum was injected into the catheter, inoculating the lungs bi-laterally with 50 μL of inoculum. Immediately after injection of the inoculum, the syringe and catheter were removed from the trachea, and the mouse was allowed to resume a normal respiration pattern. The muscle and fascia were closed over the trachea with forceps. The skin was closed using approximately 10 μL of Vet Seal (B. BRAUN MEDICAL AG, Cat. # J-299). The mice were placed in a clean litter free cage for recovery. When the mice were fully recovered and walking about, they were put individually into clean-bedded cages and housed in the post-challenge room.

Mice were monitored for 10 days post challenge on a regular basis as appropriate for any signs of distress. After 10 days, mice were sacrificed for saliva collection, lung lavages and histopathology. An IP injection of 1% pilocarpine (ALCON) (100 μG) will be administered to collect saliva samples and then the mice will be humanely euthanized. Nasal washes were performed by cannulating the trachea and slowly pumping 1 mL of PBS containing bovine serum protease inhibitors through the trachea up the nasal passage where the fluid was collected. Lung lavages were performed by repeatedly flushing the lungs with 1 mL PBS by means of a syringe connected to the cannula, which was directed towards the lung surface. Antibody titers and antibody isotypes are determined by ELISA assays (serum, saliva, and lung lavage samples). Qualitative bacteriology was performed on 100 μL aliquot of unprocessed BAL fluid. Complete dilutions from 1:1 to 1:100 were evaluated. Each dilution of BAL fluid was streaked in triplicate on Trypsin Soy agar (TSA) and Pseudomonas Isolation agar (DIFCO, Cat. #292710) (PIA) plates and incubated overnight at 37° C. Colony forming units were counted. The remaining unprocessed BAL fluid was treated with 100 mM phenylmethylsulfonyl fluoride (PMSF, SIGMA, Cat. # P-7629) and 5 mM EDTA (FISHER SCIENTIFIC, Cat. # E478-500). BAL fluid was then centrifuged for 10 minutes at 100 g at 4° C. Supernatant was removed from the pellet and filtered through a 22 μM syringe filter. The sample is stored at 70° C. until cytokine analysis could be preformed. The cell pellet was resuspended gently in 1 mL sterile PBS and a 90 μL sample of suspended cells added to 10 μL Trypan Blue. A 10 μL sample was added to a hemacytometer and cell count was performed.

TABLE 17
Colony Forming Units in BAL Fluid

| Treatment (route(s) of injection) | CFU (× 10³)/mL |
|---|---|
| CS1 (i.n.) | 0 |
| CS1 (i.p.) | 5 |
| CS1 (i.n. & i.p.) | 47 |
| Linear TT (i.n. & i.p.) | 140 |
| PBS (i.n. & i.p.) | 210 |

TABLE 18
White Blood Cell Counts in BAL Fluid

| Treatment (route(s) of injection) | Total Cells (× 10⁴)/mL |
|---|---|
| CS1 (i.n.) | 1 |
| CS1 (i.p.) | 28 |
| CS1 (i.n. & i.p.) | 4 |
| Linear TT (i.n. & i.p.) | 92 |
| PBS (i.n. & i.p.) | 85 |

These results demonstrated that the consensus sequence immunogen could be administered by various immunization routes to induce active antibody protection to Pseudomonas aeruginosa chronic lung infection. This kind of mucosal immunity may indeed be necessary to afford protection in various patient groups including but not limited to CF patients, bums patients, acute and chronic lymphoma and leukemia patients, long-term care and nursing home patients and immunosuppressed patients. The consensus sequence immunogen can also elicit an antibody response to afford protection to Pseudomonas aeruginosa infection in the general population (adult and infant population) and specialized groups at potential risk of Pseudomonas infections such as firefighters, rescue team, and medical employees.

### EXAMPLE 19

Passive Intratracheal Passive Immunization Model

CF mice received from Case Western University were acclimatized for one week before surgery. Mice were weighed the day of surgery and pre-medicated with Buprenorphine (RECKITT & COLEMAN, INC.) 0.1 mg/kg s.c. for intra-operative and post-operative analgesia. Mice were anesthetized with a cocktail of Ketamine (BIMEDA-MTC, INC.) 75 mg/kg, and Acepromazine (AYERST, INC.) 2.5 mg/kg injected i.p. Anesthetic cocktail: 0.72 ml Ketamine (BIMEDA_MTC, INC.), 0.24 ml Acepromazime (AYERST, INC.), and 8.64 ml sterile PBS were mixed into a sterile 10 ml capped vial. Anesthetic cocktail was made fresh the morning of surgery. NAIR depilatory cream (CARTER-HOMER, INC.) was applied to the throat area for 10 minutes to remove hair from the surgical site. After 10 minutes, the NAIR and hair are wiped away with 2×2 gauze (JOHNSON & JOHNSON).

Once the mouse was in surgical plane anesthesia, it was placed in dorsal recumbancy. A piece of 3/0 silk suture (ETHICON, INC.) was used to lasso the upper incisors and secure the head in a steady position. The tail was secured with masking tape. The surgical site was prepped with proviodine. A 1 cm skin incision was made on the ventral cervical surface cranial to the thoracic inlet of the trachea. The subcutaneous fat and muscle was reflected using smooth forceps and blunt dissection. Muscle and fascia was dissected from the trachea such that it could be clearly seen. A 24 g×3/4 inch venocatheter (JORVET, INC.) was used to cannulate the trachea. Caudal to the larynx, the needle was inserted bevel up into the lumen of the trachea.

After introduction of the catheter, the needle was removed, and the catheter was advanced to a point above the bifurcation of the trachea. A 1 mL syringe was filled to 200 μL with the inoculum (Pseudomonas aeruginosa agarose beads). The syringe was attached to the hub of the catheter. The hub and lumen of the catheter hold 50 μL, 100 μL of inoculum was injected into the catheter, inoculating the lungs bi-laterally with 50 μL of inoculum. Immediately after injection of the inoculum, the syringe and catheter were removed from the trachea, and the mouse was allowed to resume a normal respiration pattern. The muscle and fascia were closed over the trachea with forceps. The skin was closed using approximately 10 μL of Vet Seal (B. BRAUN MEDICAL AG, Cat. # J-299). The mice were placed in a clean litter free cage for recovery. When the mice were fully recovered and walking about, they were put individually into clean-bedded cages and housed in the post-challenge room.

Post-challenge mice were anesthetized with Halothane (precision vaporizer mode), or injectable Ketamine (BI-MEDA-MTC, INC.), HCl at 50 mg/kg i.m. prior to intranasal therapy to reduce loss of monoclonal antibody over the nares and to ease induction. A.BY/SnJ or BALB/c mice are injected intranasal (˜10 μL-12.5 μL/nare), and/or intraperitoneal (150 μL) with monoclonal antibody. Mice were monitored for 10 days post challenge on a regular basis as appropriate for any signs of distress. After 10 days, mice will be sacrificed for nasal washes, lung lavages and histopathology. An intraperitoneal injection of pilocarpine (100 μG) was administered to collect saliva samples, and then the mice were humanely euthanized. Nasal washes and lung lavages were performed by cannulating the trachea and slowly pumping 1 mL of PBS containing bovine serum protease inhibitors through the trachea up the nasal passage where the fluid was collected (nasal wash) or by repeatedly flushing the lungs with 1 ml PBS by means of a syringe connected to the cannula which was directed towards the lung surface (lung lavage). Antibody titers and antibody isotypes were determined by ELISA assays (serum, nasal, and lung lavage samples). Opsonization and anti-adhesion in vitro assays were performed on the high titer nasal washes and lung lavages to determine mucosal antibody protective efficacy.

A consensus sequence monoclonal antibody made in accordance with an embodiment of the present invention can be used as a therapeutic against acute and chronic Pseudomonas aeruginosa infections. Clinical use for a monoclonal antibody therapeutic of the present invention for Pseudomonas aeruginosa can include but is not limited to, multiple trauma, patients with serious infection, cancer patients, post-BMT patients, sever pneumonia patients, patients with septic shock, CF patients, bums and inhalation trauma patients and immunosuppressed patients with ulcers. The monoclonal antibody according to an embodiment of the present invention can also be administered as a preventative for select patients on cytotoxin chemotherapy, severely immunosuppressed bum patients, and patients with difficult to treat bloodstream infections. The monoclonal antibody therapeutic in an embodiment can be administered as an adjunct therapy with antibiotic treatment.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. An antibody produced against a peptide composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

2. The antibody of claim 1 wherein the antibody comprises a monoclonal antibody.

3. The antibody of claim 2 wherein the antibody comprises a humanized monoclonal antibody.

4. The antibody of claim 3 wherein the humanized monoclonal antibody comprises a human monoclonal antibody.

5. The antibody of claim 2 wherein the antibody comprises a mouse monoclonal antibody.

6. A cell line producing the antibody of claim 1.

7. A monoclonal antibody produced against a composition including a peptide having SEQ ID NO: 3 and a carrier molecule coupled to the peptide.

8. The monoclonal antibody of claim 7 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

9. The monoclonal antibody of claim 7 wherein the monoclonal antibody comprises a humanized monoclonal antibody.

10. The monoclonal antibody of claim 9 wherein the humanized monoclonal antibody comprises a human monoclonal antibody.

11. The monoclonal antibody of claim 7 wherein the monoclonal antibody comprises a mouse monoclonal antibody.

12. A cell line producing the monoclonal antibody of claim 7.

13. A humanized monoclonal antibody or fragment thereof that is immunoreactive with Pseudomonas aeruginosa pilus protein.

14. The humanized monoclonal antibody of claim 13 wherein the humanized monoclonal antibody comprises a human monoclonal antibody.

15. A humanized monoclonal antibody or fragment thereof that is immunoreactive with the C-terminal disulfide-linked peptide region of Pseudomonas aeruginosa pilus protein.

16. The humanized monoclonal antibody of claim 15 wherein the humanized monoclonal antibody comprises a human monoclonal antibody.

17. A humanized monoclonal antibody produced against a peptide composition including a peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

18. The humanized monoclonal antibody of claim 17 wherein the composition further includes a carrier molecule coupled to the peptide.

19. The humanized monoclonal antibody of claim 18 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

20. A cell line producing the humanized monoclonal antibody of claim 17.

21. The humanized monoclonal antibody of claim 17 wherein the humanized monoclonal antibody comprises a human monoclonal antibody.

22. A pharmaceutical agent comprising an antibody or fragment thereof produced against a composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

23. The pharmaceutical agent of claim 22 wherein the antibody comprises a monoclonal antibody.

24. The pharmaceutical agent of claim 23 wherein the monoclonal antibody comprises a humanized monoclonal antibody including a human monoclonal antibody.

25. The pharmaceutical agent of claim 23 wherein the monoclonal antibody comprises a mouse monoclonal antibody.

26. The pharmaceutical agent of claim 22 wherein the composition further includes a carrier molecule coupled to the peptide.

27. The pharmaceutical agent of claim 26 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

28. A method of producing an antibody, the method comprising the steps of:
providing a peptide composition including a peptide selected from the group consisting of SEQ ID NO: 3, SEQ ID NO: 4, SEQ ID NO: 5, SEQ ID NO: 6, SEQ ID NO: 7 and SEQ ID NO: 8; and
producing the antibody against the peptide composition.

29. The method of claim 28 wherein the antibody comprises a monoclonal antibody.

30. The method of claim 29 wherein the monoclonal antibody comprises a humanized monoclonal antibody including a human monoclonal antibody.

31. The method of claim 29 wherein the monoclonal antibody comprises a mouse monoclonal antibody.

32. The method of claim 28 wherein the peptide composition further includes a carrier molecule coupled to the peptide.

33. The method of claim 32 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

34. A method for producing a humanized monoclonal antibody, the method comprising the steps of:
providing a peptide composition including a peptide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14; and
administering to an animal the peptide to produce the humanized monoclonal antibody.

35. The method of claim 34 wherein the peptide composition further includes a carrier molecule coupled to the peptide.

36. The method of claim 35 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

37. A method of treating or preventing infection by an infectious organism including Pseudomonas aeruginosa comprising administering a pharmaceutical agent including an antibody or fragment thereof produced against a composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

38. The method of claim 37 wherein the antibody comprises a monoclonal antibody.

39. The method of claim 38 wherein the monoclonal antibody comprises a humanized monoclonal antibody including a human monoclonal antibody.

40. The method of claim 38 wherein the monoclonal antibody comprises a mouse monoclonal antibody.

41. The method of claim 37 wherein the composition further includes a carrier molecule coupled to the peptide.

42. The method of claim 41 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

43. The method of claim 37 wherein the infectious organism is further selected from the group consisting of Acinetobacter ssp., Burkholderia cepacia, Haemophilus influenza and Pasteurella multiocida.

44. A method of treating or immunizing a subject against infection by an infectious agent including Pseudomonas aeruginosa comprising administering a pharmaceutical agent including an antibody or fragment thereof produced against a composition including a peptide selected from the group consisting of SEQ ID NO: 3, SEQ ID NO: 4, SEQ ID NO: 5, SEQ ID NO: 6, SEQ ID NO: 7 and SEQ ID NO: 8.

45. The method of claim 44 wherein the antibody comprises a monoclonal antibody.

46. The method of claim 44 wherein the monoclonal antibody comprises a humanized monoclonal antibody including a human monoclonal antibody.

47. The method of claim 44 wherein the monoclonal antibody comprises a mouse monoclonal antibody.

48. The method of claim 44 wherein the composition further includes a carrier molecule coupled to the peptide.

49. The method of claim 48 wherein the carrier molecule is selected from the group consisting of a carrier protein, a carrier glycoprotein, a carrier carbohydrate, tetanus toxoid/toxin, diphtheria toxoid/toxin, pseudomonas mutant carrier, bacteria outer membrane proteins, crystalline bacterial cell surface layers, endotoxins, exotoxins, serum albumin, gamma globulin, keyhole limpet hemocyanin, recombinant, exotoxin A, LT toxin, Cholera B toxin, Klebsiella pneumoniae OmpA, Bacterial flagella, Clostridium difficile recombinant toxin A, peptide dendrimers, pan DR epitope (PADRE), Commensal bacteria, Phage, peptides attached to recombinant IgG1, carrier sub-units thereof and combinations thereof.

50. The method of claim 44 wherein the infectious agent is further selected from the group consisting of Acinetobacter ssp., Burkholderia cepacia, Haemophilus influenza and Pasteurella multiocida.

51. A purified antibody or fragment thereof that binds to an epitope in a Pseudomonas aeruginosa pilin peptide or variant thereof selected from the group consisting of SEQ ID NO: 3, SEQ IDS NO: 4, SEQ ID NO: 5, SEQ ID NO: 6, SEQ ID NO: 7 and SEQ ID NO: 8.

52. The purified antibody of claim 51 wherein the purified antibody or fragment thereof can inhibit binding of an infectious agent including Pseudomonas aeruginosa to a host cell.

53. The purified antibody of claim 52 wherein the infectious agent is further selected from the group consisting of Acinetobacter ssp., Burkholderia cepacia, Haemophilus influenza and Pasteurella multiocida.

54. A humanized antibody or fragment thereof that binds to an epitope in a Pseudomonas aeruginosa pilin peptide or variant thereof selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 and SEQ ID NO: 14.

55. The purified antibody of claim 54 wherein the purified antibody or fragment thereof can inhibit binding of an infectious agent including Pseudomonas aeruginosa to a host cell.

56. The purified antibody of claim 54 wherein the infectious agent is further selected from the group consisting of Acinetobacter ssp., Burkholderia cepacia, Haemophilus influenza and Pasteurella multiocida.


SEQUENCE LISTING (14 sequences)

SEQ ID NO: 1 - protein, 17 aa, Artificial Sequence
  Synthetic peptide
KCTSDQDEQFIPKGCSK

SEQ ID NO: 2 - protein, 17 aa, Artificial Sequence
  Synthetic peptide
ACKSTQDPMFTPKGCDN

SEQ ID NO: 3 - protein, 17 aa, Artificial Sequence
  Synthetic peptide
KCKSDQDPQFIPKGCSK

SEQ ID NO: 4 - protein, 17 aa, Artificial Sequence
  Synthetic peptide
KCTSTQDPQFIPKGCSK

SEQ ID NO: 5 - protein, 17 aa, Artificial Sequence
  Synthetic peptide
KCTSTQDPQFTPKGCSK

SEQ ID NO: 6 - protein, 17 aa, Artificial Sequence
  Synthetic peptide
KCKSTQDEMFTPKGCSK

SEQ ID NO: 7 - protein, 17 aa, Artificial Sequence
  Synthetic peptide
KCTSDQDAQFIPKGCSK

SEQ ID NO: 8 - protein, 17 aa, Artificial Sequence
  Synthetic peptide
KCTSDQDPQFIPKGCSK

SEQ ID NO: 9 - protein, 17 aa, Artificial Sequence
  Synthetic peptide
TCTSTQEEMFIPKGCNK

SEQ ID NO: 10 - protein, 17 aa, Artificial Sequence
  Synthetic peptide
ACTSNADNKYLPKTCQT

SEQ ID NO: 11 - protein, 17 aa, Artificial Sequence
  Synthetic peptide
SCATTVDAKFRPNGCTD

SEQ ID NO: 12 - protein, 22 aa, Artificial Sequence
  Synthetic peptide
NCKITKTPTAWKPNYAPANCPK

SEQ ID NO: 13 - protein, 22 aa, Artificial Sequence
  Synthetic peptide
TCGITGSPTNWKANYAPANCPK

SEQ ID NO: 14 - protein, 22 aa, Artificial Sequence
  Synthetic peptide
GCSISSTPANWKPNYAPSNCPK